(19) United States
(12) Patent Application Publication
(10) Pub. No.: US 2026/0083575 A1
(43) Pub. Date: Mar. 26, 2026

(54) DEVICES AND METHODS FOR USE DURING KNEE REPLACEMENT SURGERY

(71) Applicant: Jae S Son, Rolling Hills Estates, CA (US)

(72) Inventor: Jae S Son, Rolling Hills Estates, CA (US)

(21) Appl. No.: 19/404,197

(22) Filed: Dec. 1, 2025

Related U.S. Application Data
(63) Continuation-in-part of application No. 18/241,937, filed on Sep. 4, 2023, now Pat. No. 12,485,025.

(51) Int. Cl. A61F 2/46; A61B 17/00; A61B 17/02; A61B 90/00; A61F 2/38

(52) U.S. Cl. CPC A61F 2/4657; A61B 17/0218; A61B 2017/00557; A61B 2017/0225; A61B 2090/064; A61F 2/38; A61F 2002/4666

(57) ABSTRACT

A knee alignment measurement tool for use during a total knee replacement surgery may include the following main components: a sensor assembly, a display unit configured to power up, operatively control the individual sensors of the sensor assembly, and display the readings therefrom, and a flexible gooseneck cable operatively connecting the sensor assembly to the display unit. The sensor assembly may include a rigid base supporting a circuit board, a left sensor and a right sensor positioned adjacent to the left center, and a cover sealed to the rigid base to enclose the left sensor and the right sensor, wherein the cover does not abut the rigid base during the compression of the sensor assembly.

### CROSS-REFERENCE DATA

This patent application is a continuation-in-part of a co-pending U.S. patent application Ser. No. 18/241,937 filed on 4 Sep. 2023 with the same title, now U.S. Pat. No. 12,485,025, which is incorporated herein in its entirety by reference.

### BACKGROUND

Without limiting the scope of the invention, its background is described in connection with various devices and methods that may be used during knee replacement surgery. More particularly, the invention describes devices and methods for measuring orthotic fit, as well as the balance and consistency of forces on the adjacent condyles through a range of knee motion.

Knee arthroplasty, or knee replacement surgery, has long aimed to restore pain-free knee functionality. Knee replacement surgery is a complex procedure designed to alleviate pain and restore functionality to a knee joint damaged by injury or degenerative conditions like osteoarthritis. It involves several critical steps, with a primary focus on achieving a balance of forces on the knee prosthesis across the entire range of motion, ensuring longevity and a pain-free operation of the artificial knee.

The balance of forces is achieved during surgery by meticulous attention to detail and precise alignment of the artificial components. The surgeon will assess the ligaments, tendons, and other soft tissues to ensure they are properly tensioned. Imbalanced positioning of the knee may cause complications such as instability or uneven wear on the prosthesis.

The artificial knee components are implanted once the joint is prepared and balanced. This typically involves fixing a metal femoral component to the end of the thigh bone and a tibial component to the top of the shin bone, with a plastic spacer in between to mimic the function of natural cartilage. If necessary, the patella (kneecap) may also be resurfaced.

After implantation, the surgeon must meticulously check the range of motion and the alignment of the knee, making any necessary adjustments to ensure that the forces are evenly distributed across the prosthesis. Achieving this balance minimizes stress on the implant and surrounding tissues, which is essential for the longevity and pain-free operation of the artificial knee.

Achieving balance in total knee replacement surgery involves a range of tools and techniques, each with its own set of detriments and limitations. Alignment guides assist in proper positioning but may face challenges in cases of unusual patient anatomy. Trial implants are crucial for fine-tuning balance but might not perfectly predict outcomes with actual implants. Computer-assisted surgery systems enhance precision but require specialized equipment and training. Gap balancing techniques adjust soft tissue tension but demand surgical expertise and can lead to over-correction or under-correction. Sensor-guided implants offer real-time feedback but are relatively new, with evolving long-term outcomes. Intraoperative imaging like X-rays or fluoroscopy confirms alignment but exposes it to radiation and may not capture soft tissue tension comprehensively. Patient-specific instrumentation relies on pre-operative imaging and may not adapt to intraoperative changes.

The need, therefore, exists for better tools to be used during knee replacement surgery, aimed at achieving a balance of forces and proper operation of a knee prosthesis for a wide variety of patients.

As part of the problem, there is a need for complete parallelism of the cuts on the tibia and femur bones. Achieving this parallelism is essential for the longevity and functionality of the replacement knee. Modern cutting tools, such as computer-assisted navigation systems and robotic-assisted surgery, have significantly improved the precision of these cuts. They provide real-time feedback to the surgeon, enabling precise alignment and ensuring that the bone cuts are parallel, which is critical for optimal implant placement. However, limitations still exist with the current approach. These tools can be expensive and require specialized training, making them less accessible to some medical facilities. Additionally, patient-specific factors and anatomical variations may not always be fully accounted for, necessitating the need for even more advanced and adaptable tools in the future to achieve the highest level of precision and parallelism in bone cuts during knee replacement surgery. Developing new tools that combine the advantages of modern technology with affordability, adaptability, and ease of use will be crucial for further enhancing surgical outcomes in this field.

### SUMMARY

Accordingly, it is an object of the present invention to overcome these and other drawbacks of the prior art by providing a novel precision knee alignment tool configured to provide feedback during knee replacement surgery on compression forces between components of a knee replacement prosthesis.

It is another object of the present invention to provide a knee alignment tool configured to provide feedback on the balance of forces between a left condyle and a right condyle of the knee prosthesis.

It is a further object of the present invention to provide a novel knee alignment tool in which compression forces are measured accurately throughout the range of motion of the knee prosthesis.

It is yet a further object of the present invention to provide a novel knee alignment tool configured to cause compression forces to affect only the respective pressure sensor readings and not be distorted by interference from the housing containing the pressure sensors.

A knee alignment measurement tool for use during a total knee replacement surgery may include the following main components: a sensor assembly, a display unit configured to power up, operatively control the individual sensors of the sensor assembly, and display the readings therefrom, and a flexible cable operatively connecting the sensor assembly to the display unit. The sensor assembly may include a rigid base supporting a circuit board, a left sensor and a right sensor positioned adjacent to the left center, and a cover sealed to the rigid base to enclose the left sensor and the right sensor, wherein the cover does not abut the rigid base during the compression of the sensor assembly.

In embodiments, the rigid base of the sensor assembly may further feature a side wall surrounding the left sensor and the right sensor. The cover may be sized to fit inside the side wall of the rigid base with a predefined gap in between, which may be sealed with a compliant adhesive.

The cover of the sensor assembly may contain at least one thicker portion surrounded by at least one thinner portion to allow the thicker portion to transmit compression force applied on top thereof without affecting the remaining areas of the cover.

In further embodiments, the cover of the sensor assembly may include a first thicker portion surrounded by a first thinner portion and located above the left sensor of the sensor assembly. Furthermore, the cover of the sensor assembly may also include a second thicker portion surrounded by a second thinner portion and located above the right sensor of the sensor assembly. This design may cause the compression of the left sensor through the cover of the sensor assembly, which does not affect the compression of the right sensor thereof.

In further embodiments, the thicker portions extend above the top surface of the cover and below the bottom surface thereof, thereby defining the portions of the cover that transmit compression forces to the sensors of the sensor assembly during knee replacement surgery.

A gooseneck cable comprises a jacket containing a flex interconnect and a malleable wire configured to allow for diverse orientation and positioning of the display unit during the knee replacement surgery. The flex interconnect comprises a plurality of electrical connections between the sensor assembly and the display unit. The malleable wire may be a copper wire with a hemispherical cross-sectional shape. The malleable wire may extend from the sensor assembly through the jacket of the gooseneck cable into a loop retained in the display unit, and back through the jacket of the gooseneck cable to the sensor assembly, thereby facilitating the use of the gooseneck cable as a pull tail of the sensor assembly.

A test and calibration apparatus is described to be used for calibrating the sensor assembly of the knee alignment device. The test apparatus may comprise a kinematic mount with provisions to accept different sensor assemblies in a consistent manner and apply the forces at various predetermined positions on the sensor assembly using a loading plate.

A calibration algorithm is described to be used during the calibration process in order to process various locations and levels of forces. The forces are applied using the loading plate in different predetermined positions of the sensor assembly 110. The calibration algorithm weighs the force that's being applied at the center more heavily than the forces applied over the sensing elements when the load is not balanced.

A novel exoskeleton-type stiffness measuring mechanism is described to be used along with the knee alignment device. The stiffness measurement device may comprise a pair of parallel linear actuators, a pair of rotating joints, at least one of them is equipped with a locking button, and a leg attachment fastener, such as a Velcro strap configured to be wrapped about the leg of the patient so as to mount the stiffness measuring mechanism thereon. In use during surgery, the stiffness measuring mechanism may be configured to apply a controlled amount of deflection by extending one or both linear actuators to measure the stiffness of the knee joint, while the knee alignment device facilitates the force measurement between the knee joint.

A force-sensing glove is described as comprising a plurality of force-sensing components and a built-in inertial measurement unit, which may be used to measure displacement while the surgeon applies a force to the knee joint. The force-sensing glove may be configured to measure both force and displacement during manual deflections of the knee joint by the surgeon, which allows an automated calculation of the knee joint stiffness.

A dual-blade cutting saw is described, having a pair of parallel blades separated by a replaceable spacer configured to change the distance between the blades. Once the bones of the patient are positioned and retained in a desired final alignment, the novel saw may be used to provide both cuts of the respective bones at the same time, thereby facilitating proper positioning of the knee prosthesis and improving the longevity of the knee prosthesis' pain-free function over the full range of motion.

The present invention adds height-adjustment and distance-measurement capabilities to the existing knee alignment measurement tool used during knee replacement surgery. The core instrument stacks three main elements along the load path between the femur and tibia: a femur-facing insert that maintains contact with the femoral component, a knee alignment measurement tool beneath it, and a shim placed between them and around a central post of the tool. By selecting or adjusting the shim, the surgeon can change the separation distance between the femur-facing insert and the measurement tool, and therefore finely adjust the spacing between the femur and tibia during alignment and balancing.

Typically, this spacing is changed using a set of interchangeable rigid shims of different thicknesses. The surgeon inserts one shim at a time between the femur-facing insert and the knee alignment tool; the thickness of the chosen shim directly defines the distance between these components and thus the joint gap. In one version, the surgeon needs to remove the assembly from the knee to replace the shim, which is time-consuming, so another version allows the shim to be removed and another shim to be inserted without removing the entire assembly. In this specification, a more advanced version allows the spacing to be changed continuously using an inflatable shim. This inflatable shim may be positioned in the middle or on one side of the stack, having the femur-facing insert and the knee alignment tool. The inflatable shim may contain an inflatable bladder and a distance sensor. As the bladder is inflated or deflated through an inflation port, the overall height of the stack changes in a measurable way that corresponds to a change in the distance between the femur and tibia.

The inflatable shim may be mounted on a rigid base that can be detached from the bladder. This allows the bladder to be replaced after each surgery while reusing the more durable rigid base structure. The shim is designed to be used in several stack configurations: the knee alignment tool may be positioned between the shim and the femur-facing insert, and the inflatable shim may partially surround a central post extending from the measurement tool. This central post helps mechanically locate and constrain the shim relative to the tool and the insert so that inflation produces predictable and repeatable changes in gap height.

The knee alignment tool itself, as described above, includes a sensor assembly and a display unit. The sensor assembly has a rigid base that supports left and right force sensors side by side, covered by a somewhat compliant material that routes compressive forces separately to each sensor. The display unit powers and controls the left sensor, the right sensor, and the shim's distance sensor, and presents force and distance readings to the surgeon in real time. In use, the system can simultaneously report the total compressive force between the components of the knee prosthesis, the balance of force between the medial (left) and lateral (right) condyles, and the current distance between the femur and tibia. The contactless distance sensor can be, for example, a capacitive sensor or a lidar-based sensor using pulsed laser light.

In some embodiments, the inflatable bladder includes two distinct balloons in various regions of the sensor assembly, one aligned with the left sensor or a group of left sensors and the other with the right sensor or a group of right sensors. When this dual-balloon shim is inserted between the knee alignment tool and the femur-facing insert, each balloon is shaped and positioned to sit directly over its corresponding sensor. As the bladder is inflated, the left balloon transmits force to the left sensor and the right balloon transmits force to the right sensor, preserving independent, side-specific measurements while the joint space is adjusted. The range of added height from inflation can be, for example, on the order of about 1 mm to 8 mm, giving the surgeon fine control over joint gap while maintaining accurate and balanced force readings.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 is a perspective view of the knee alignment device 100,

FIG. 2 is an exploded view of the sensor assembly 110 of the device 100,

FIG. 3 shows a close-up perspective view of the components of the sensor assembly 110,

FIG. 4 is a more detailed perspective view of the sensor assembly 110,

FIG. 5 is a close-up cross-sectional view of the sensor assembly 110,

FIG. 6 is a close-up cross-sectional perspective view of the sensor assembly 110,

FIG. 7 is a cross-sectional view of the device 100 showing the details of the gooseneck cable 190,

FIG. 8 is a top perspective view of the center post of the sensor assembly 110,

FIG. 9 is a bottom perspective view of the same,

FIG. 10 is a partial perspective view of the joint between the gooseneck cable and the sensor assembly of the invention,

FIG. 11 is a partial cross-sectional perspective view of the same,

FIG. 12 shows a diagram of the knee alignment device in use,

FIG. 13 shows a diagram of the knee alignment device positioned over a calibration instrument,

FIG. 14 shows a schematic location of force application during calibration of the knee alignment device 100,

FIG. 15 shows an exemplary algorithm used during the calibration procedure,

FIG. 16 is a schematic view of the knee joint stiffness measurement device of the present invention,

FIG. 17 is a cross-sectional view showing an attachment of the stiffness measurement device to a leg during surgery,

FIGS. 18A, 18B, and 18C show the stiffness measurement device of the invention in various use positions,

FIG. 19 shows a novel glove of the invention configured to measure force during knee replacement surgery,

FIG. 20 shows various alignment views for the bones during orthopedic surgery,

FIG. 21 shows successive cuts made during orthopedic surgery,

FIG. 22 shows a novel dual-blade saw that may be used during knee replacement surgery,

FIG. 23 shows a general perspective view of the knee alignment measurement instrument of one embodiment with the femoral contacting insert on top and a shim in between,

FIG. 24 shows an exploded perspective view of the knee alignment measurement instrument of FIG. 23,

FIG. 25 shows a top view of the same,

FIG. 26 shows a cross-sectional view of the same along the plane indicated by the line 26-26 in FIG. 25,

FIG. 27 shows a perspective view of another embodiment of the knee alignment tool of the present invention with the femoral contacting insert on top and a shim in between,

FIG. 28 shows an exploded perspective view of the knee alignment measurement instrument using the knee alignment measurement tool of FIG. 27,

FIG. 29 shows a top view of the same,

FIG. 30 shows a cross-sectional view of the same along the plane indicated by the line 30-30 in FIG. 29,

FIG. 31 shows a general illustration of an inflatable shim,

FIG. 32 shows a schematic depiction showing a stack of knee alignment measuring devices using the inflatable shim,

FIG. 33 shows an exploded perspective view with the inflatable shim placed between the femur-facing insert and the knee alignment measuring tool,

FIG. 34 shows an exploded perspective view with the inflatable shim placed below the knee alignment measuring tool,

FIG. 35 shows an exploded perspective view of another embodiment of the invention having the inflatable shim placed below the knee alignment measuring tool,

FIG. 36 shows a top view of the instrument of FIG. 35,

FIG. 37 shows a cross-sectional view of the same along the plane indicated by the line 37-37 in FIG. 36,

FIG. 38 shows an exploded perspective view of another embodiment of the knee alignment measurement instrument of the invention,

FIG. 39 shows a top view of the same,

FIG. 40 shows a cross-sectional view of the same along the plane indicated by the line 40-40 in FIG. 39, and

FIG. 41 shows an alternative sensor configuration used for thermal compensation.

### DETAILED DESCRIPTION OF PREFERRED EMBODIMENTS OF THE INVENTION

The following description sets forth various examples along with specific details to provide a thorough understanding of the claimed subject matter. It will be understood by those skilled in the art, however, that claimed subject matter may be practiced without one or more of the specific details disclosed herein. Further, in some circumstances, well-known methods, procedures, systems, components and/or circuits have not been described in detail in order to avoid unnecessarily obscuring the claimed subject matter. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein and illustrated in the figures, can be arranged, substituted, combined, and designed in a wide variety of different configurations, all of which are explicitly contemplated and make part of this disclosure.

During a knee replacement surgery, the surgeon removes the damaged parts of the knee joint, including the cartilage and bone, and replaces them with artificial components made of metal, plastic, or ceramic. The goal is to create a new joint that functions like a normal knee joint, with a smooth range of motion and stability. More specifically, the surgeon initially prepares the patient's bone(s) to receive the orthopedic prosthesis. To do so, the surgeon may resect a portion of the patient's proximal tibia to which the tibia tray will be attached, and a portion of the patient's distal femur to which the femoral component will be attached. During such procedures, the surgeon may attempt to balance or otherwise distribute the joint forces of the patient's joint in order to produce joint motion that is similar to the motion of a natural joint. To do so, the surgeon may use surgical experience and manually “feel” for the appropriate joint force balance. Additionally, or alternatively, the orthopedic surgeon may use surgical instruments, such as a ligament balancer in the case of a knee replacement procedure, to assist in the balancing or distributing of joint forces.

The amount of force and tension that a knee joint can withstand during a knee replacement surgery depends on various factors, such as the patient's age, weight, activity level, and overall health. Generally, surgeons aim to restore the normal biomechanics of the knee joint during surgery, which involves applying appropriate forces and tensions to the joint. The forces and tensions applied during surgery can also vary depending on the surgical technique and the type of implant used. Generally, the forces applied to the knee joint during surgery range from 120 to 250 Newtons, depending on the specific surgical approach and implant used.

One of the tools that may be used during this part of the surgery is a knee alignment tool. The precision knee alignment tool is used during surgery to be placed between a metal femoral component at the end of the thigh bone and a tibial component to the top of the tibia bone. Prior art devices describe a variety of such tools; for example, in the U.S. Pat. Nos. 5,197,488; 5,360,016; 5,456,724; 5,470,354; 5,656,785; 7,195,465; 7,412,897; 7,575,602; 7,632,283; 7591854; 8551023; 10098761; 10206792; 11051955; and 11096801. These tools generally have a sensor assembly including two lobes, each containing a sensor configured to measure the force between the individual portions of the knee replacement joint. A common feature of many of these designs is the presence of a clam-shell housing containing the sensor portion of the device. The top portion of the “clamshell” is positioned over the bottom portion thereof such that the edge of the sidewall of the top portion abuts the edge of the side wall of the bottom portion. This causes a limitation of performance in that the load path goes through the injection molded housing. In other words, compression of the top portion of the clamshell housing interferes with the bottom portion. Dimensional variability of the injection molded parts is usually excessive to provide consistent performance, in that one part of the housing affects the other part and distorts the force transmitted to the actual sensor located within the housing itself.

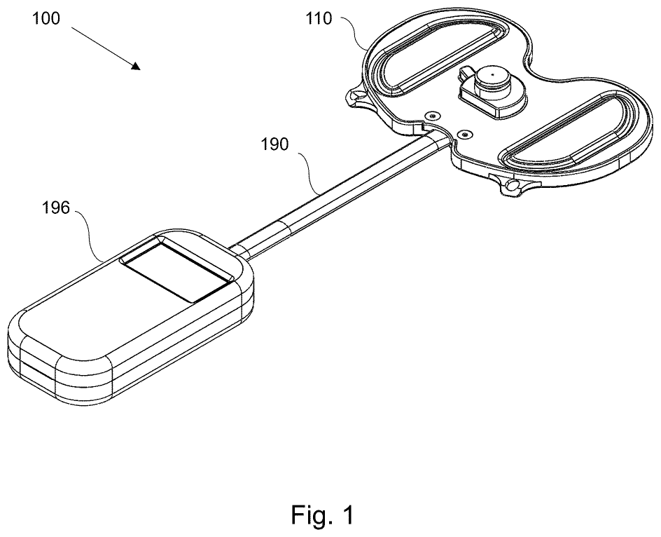

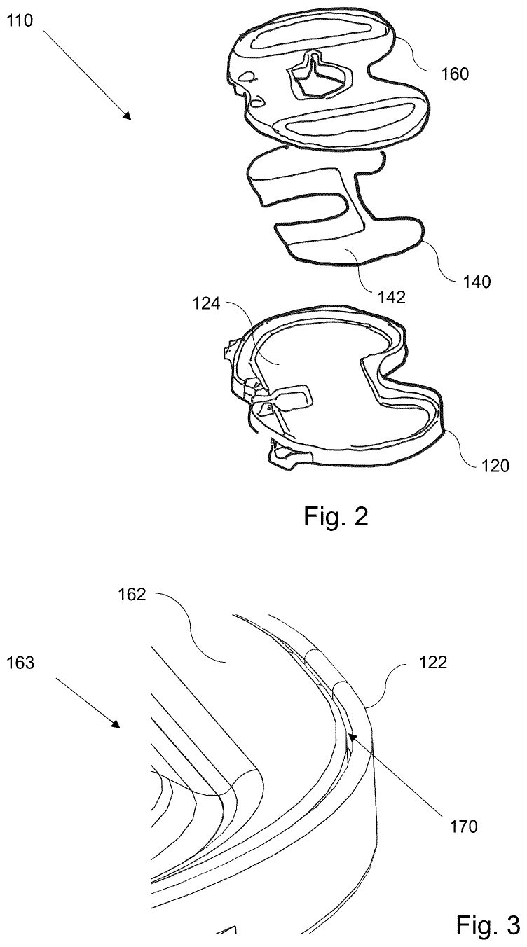

The first described part of the present invention is a novel precision knee alignment tool 100 shown in general in FIG. 1, which comprises a sensor assembly 110 connected to a display unit 196 by a flexible gooseneck cable 190. Additional drawings showing different elements of the design of the knee alignment tool are seen in FIGS. 2-6 accompanying this disclosure.

The main innovative feature is the absence of the clam-shell rigid housing that limits the extent of the performance of the tool. Instead, a precisely machined metal or rigid plastic base 120 is used to house all other sensing and cover components assembled on top thereof in a “floating” manner so as to not interfere with other components. The performance of the sensor portion of the device, in this case, is not limited by the rigid features and elements of the base 120.

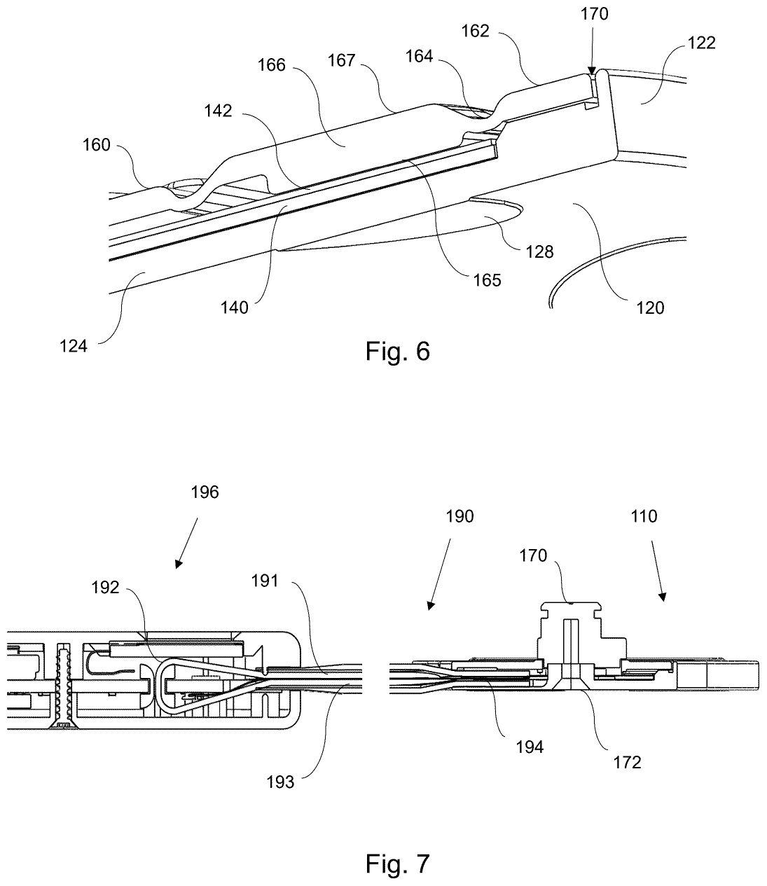

A better appreciation of this design may be gained with reference to FIG. 2, which shows an exploded view of the main elements of the sensor assembly 110. The rigid base 120 may have a flat dual-kidney-shaped depression 124 with a side wall 122 surrounding thereof and forming a cavity for positioning a sensor subassembly 140 and the sensor cover 160 therein. The rigid base 120 may be made from a rigid material such as plastic or metal. In other embodiments, the rigid base may have a metal plate secured on top of the flat depression 124 and configured to serve as a supporting member for the sensors of the sensor assembly 110. A sensor subassembly 140, in turn, may include a “floating” flexible printed circuit board 142 containing a pair of a left sensor and a right sensor positioned thereon. Kapton or another flexible substrate of the flexible PCB 140 may be selected such that the performance of the left pressure sensor is not affected by compression and the performance of the right pressure sensor. Both sensors may reside on a precisely machined flat surface 124 of the base 120 (see FIG. 6). The sensor subassembly 140 may be positioned on top of the base 120 in such a manner that the left sensor is mechanically isolated from the right sensor and compression of each sensor may be registered by these sensors individually, without cross-interference.

To further assist in de-coupling one sensor from another, a convoluted cover 160 may be used to protect the sensor subassembly above thereof. Several features of the cover 160 design may be helpful in achieving this objective. First, the cover 160 may be dimensioned to fit inside the side wall of the base with a small gap 170 between the base wall 122 and the periphery 162 of the cover 160 (see FIGS. 3 and 6). This small gap may be filled with a flexible and highly compliant adhesive (for example, Nusil Med-1 silicone adhesive manufactured by NuSil Technology LLC, Carpinteria, CA) to seal the internal cavity of the device and prevent moisture ingress from outside. The extent of adhesive compliance may be selected to be sufficiently high so as to not interfere with a small movement of the cover 160 relative to the wall 122 over a range of compression forces of up to 250 N.

Secondly, the cover 160 may be made with thicker sections and thinner sections as described below. A pair of thicker sensor cover portions 166 may be made to be positioned over the corresponding left and right sensors to accurately transmit the pressure thereon. Each thicker sensor cover portion 166 may be molded to have flat parallel top and bottom surfaces so as to facilitate compression of the sensor therethrough. The thickness and the Young modulus of that portion may be selected to be stiff enough not to change its shape under a maximum load expected to be applied (such as up to 250N, or even higher for a suitable safety margin, such as up to 450N), while, on the other side, being soft and flexible enough to transmit compression without imparting any forces on the adjacent sensor.

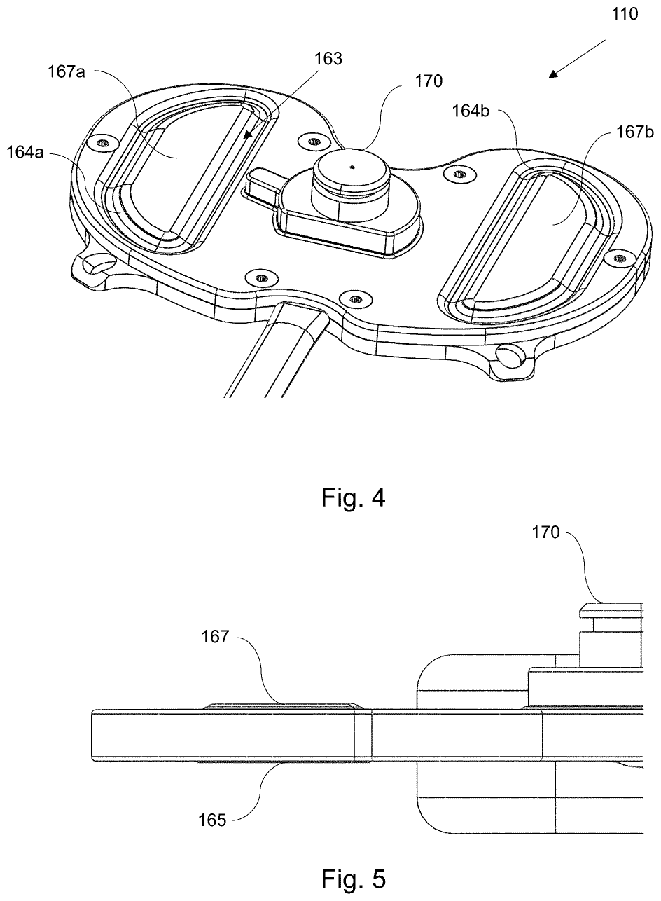

In embodiments, the cover 160 of the sensor assembly 110 may include a first thicker portion 167a surrounded by a first thinner portion 164a and located above the left sensor of the sensor assembly 110. The cover 160 of the sensor assembly 110 may further feature a second thicker portion 167b surrounded by a second thinner portion 164b and located above the right sensor of the sensor assembly. This arrangement may be used to ensure that compression of the left sensor through the cover 160 of the sensor assembly 110 does not affect compression of the right sensor thereof, see FIG. 4.

A thinner flexible portion 164 may surround the thicker stiff portion 167 so as to act as a living hinge around thereof and further mechanically isolate one sensor from the other. In other words, a thinner wall 164 of cover 160 will absorb any small motion resulting from the compression force over the corresponding sensor and will not cause any distortion of the position of cover 160 over the adjacent pressure sensor, therefore facilitating its independent measurement of the compression forces applied thereto. Transition 163 from the thicker portion to the thinner portion may be gradual or stepwise, as seen in examples in FIGS. 3 and 4.

Finally, a thicker yet outer periphery portion 162 may be helpful to attach the cover 160 to the base 120 in a more reliable manner via an adhesive or by other suitable means and techniques.

In one example, medical-grade polypropylene family of materials may be selected to be used as a biocompatible material for cover 160 with Young's modulus in a range from about 1,325 MPa to about 2,000 MPa, such as at least 1,325 MPa, at least 1,500 MPa, at least 1,750 MPa, or up to 2,000 MPa. The thickness of the thinner portion 164 may range from about 0.4 mm to about 0.6 mm, such as at least 0.4 mm, at least 0.45 mm, at least 0.5 mm, at least 0.55 mm, or up to 0.6 mm. This range may vary depending on the material and manufacturing techniques. The lower end of the range is limited by selecting a reliable manufacturing technique that can be used to produce a thin enough portion 164 without failure. The high end of the range is defined by a thickness that still allows the unrestricted flexing of the thinner portion 164 as described above. The range of thickness for a thicker portion 167 made from polypropylene may be in a range from about 1.5 mm to about 3 mm, such as at least 1.5 mm, at least 2 mm, at least 2.5 mm, or up to 3 mm. The lower end of the range is guided by the requirement of the thicker portion 167 to be stiff enough to not allow for any material movement under compression of up to 250 N, or even up to 450 N with a suitable safety margin. The upper end of the thickness is limited by the overall thickness of the sensor assembly 110. In the case of the industry standard overall thickness of the sensor assembly 110 of 4.1 mm, the upper end of the range for the thickness of the thicker portion 167 may be about 3 mm so as to allow sufficient room for other components of the sensor assembly 110 to be stacked up with the cover 160 and not exceed the allowable thickness of the device.

Importantly, the thinning of the cover 160 wall along the area 164, that surrounds each sensor allows the cover 160 portion 166 to be the only component that makes contact with the underlying corresponding sensor and transmits the compression force encountered by the knee joint. Therefore, the floating design of the thicker portions 167a and 167b and the mechanical isolation of each pressure sensor leads to a better and more independent force measurement by each sensor, which is critical during the checking of the balance of the knee joint throughout the range of knee motion.

To further ensure that only portion 166 is in contact with both the loading element on top of the device and the pressure sensor located underneath the cover 160, the top oval- or kidney-faced faces 167a and 167b of the cover portion 166 may be raised above other top surfaces of the adjacent structures, while the lower oval—or kidney-shaped face 165 may be lowered below other structures of the cover 160—see FIGS. 4, 5 and 6. The thicker portions 166, therefore, extend above the top surface of the cover 160 and below a bottom surface thereof. This design assures a force transmission to be concentrated only on the thicker portions 166 and not over the entire top surface of the cover 160. Thicker portions 166 may, therefore, flex and move toward and away from the base 120 independently of the rest of the cover 160 over the range of motion expected to independently transmit compression forces during the knee replacement surgery. No force is transmitted to the sidewall 122, which may be made to end below the face 167 of the cover 160. To further isolate the compression area to the area of device 100 corresponding to the sensor location, the base 120 may have a pair of feet 128 protruding downward and away from the base 120, each shaped similarly to the face 167 of the thicker portions 166 and located under each of the two corresponding right and left sensors of device 100. This allows the entire stack-up of the sensor assembly 110 (from the top thicker portion 166—to the sensor—to the base 120 with the protruding feet 128) to concentrate force transmission only on the area of the respective sensors.

Gooseneck malleable cable 190 is seen in a cross-section view in FIG. 7. The gooseneck cable 190 is an assembly comprising a medical-grade outer jacket containing a flex interconnect with a plurality of electrical connections between the sensor assembly and the electronics in the display unit 196, and a malleable copper wire 191 that may be, in some examples, hemispherical in cross-sectional shape. The copper wire 191 may extend from the sensor assembly 110 through the jacket of the gooseneck cable to the display unit 196. It can then form a loop 192 around the printed circuit board of the display board and then be fed as wire 193 through the outer jacket (wire 192) along with the flex interconnect. The copper wire 193 may be clamped back at the sensor assembly 110 at clamp 194.

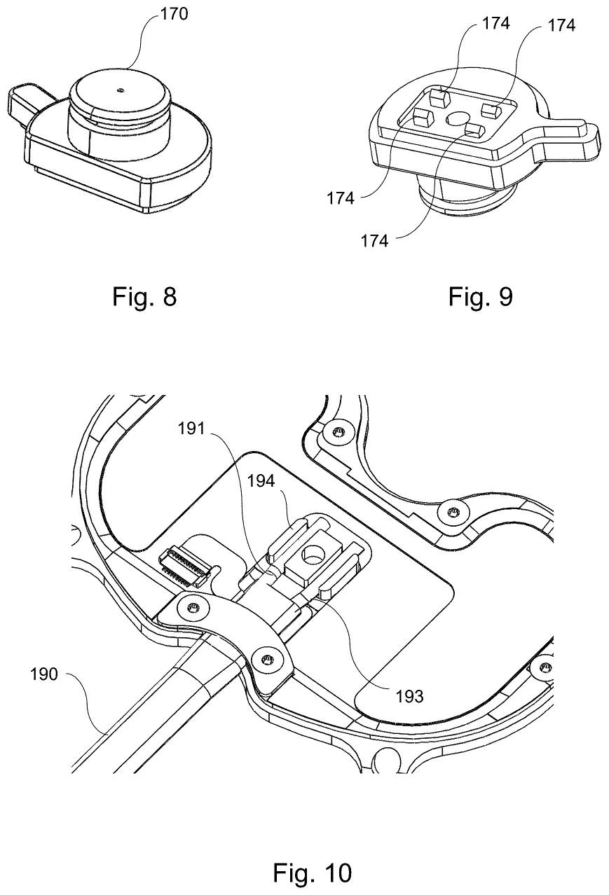

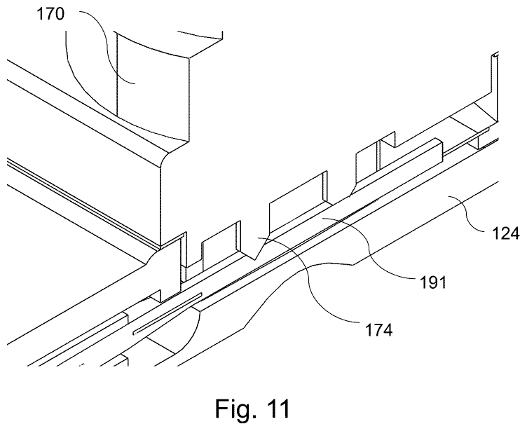

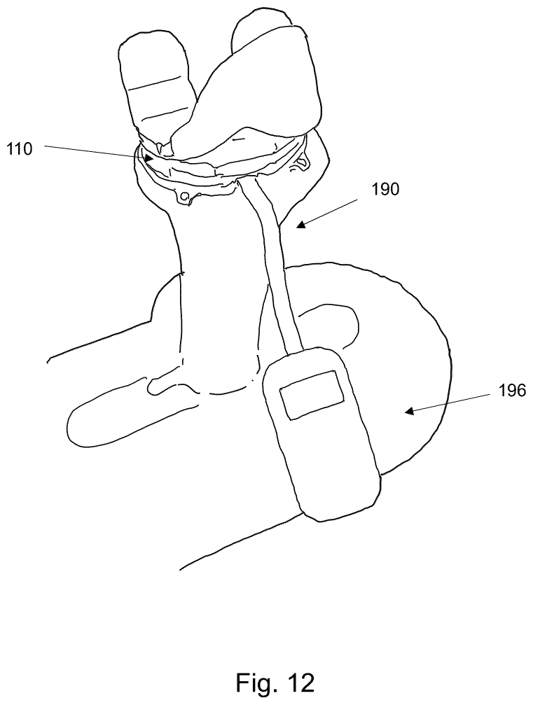

FIGS. 8 and 9 show the top and the respective bottom views of the center post 170. One or more claws 174 may be positioned on the bottom surface thereof such that when the center post is assembled onto the sensor assembly 110 and retained by a center screw 172, the claws 174 bite into the soft copper wires 191 and 193 against the clamp base 194 (see FIG. 4 as well as FIG. 10, center post 170 is removed) in order to retain the gooseneck cable 190 together with the structural base 120 of the sensor assembly 110. A close-up cross-sectional view of the cable assembly 110 at the location of the copper wires 191 and 193 is seen in greater detail in FIG. 11.

The presence of the copper wire helps the cable assembly 190 to have a malleable behavior, which allows for diverse orientation and positioning of the display unit 196 at different points during the surgery and regardless of the position of the sensor assembly 110, as seen better in FIG. 12. Independent attachment of the malleable wire to both the sensor assembly 110 as well as to the display unit 196 not only increases the pull strength of the cable 190 and reliability of a fragile flex interconnect contained therein; it also facilitates the use of the gooseneck cable 190 as a pull tail of the sensor assembly 110 during use.

The display unit 196 may include a source of power (a battery) and be configured to electrically power up the sensors of the sensor assembly 110. The display unit 196 may contain further control electronics to operate the sensors and a display to show their respective readings to the surgeon during the orthopedic surgery.

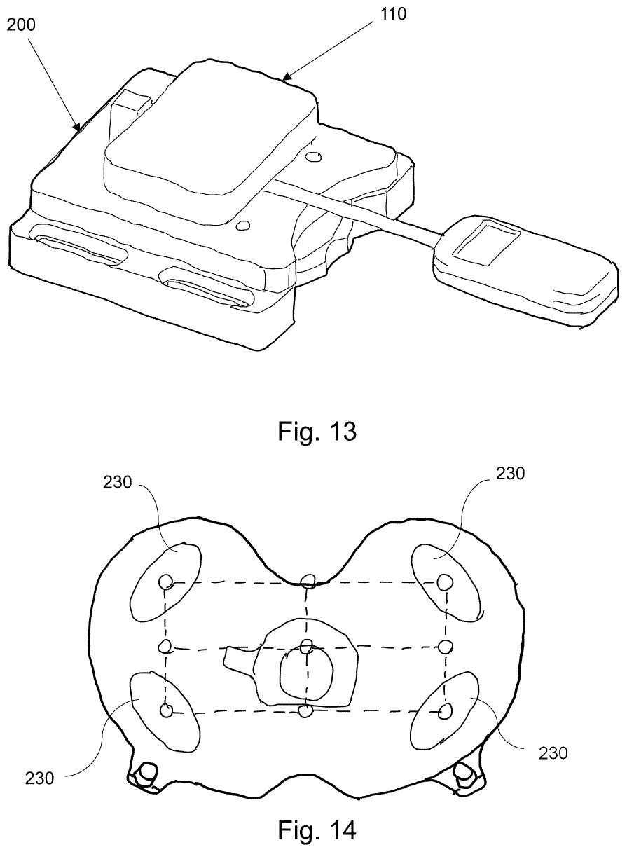

In addition to providing superior performance based on the design of the device outlined above, individual calibration may be used to further improve the performance of the tool of the present invention. A calibration device 200 and methods described below and shown in FIGS. 13-15 may provide an instrument suitable for performing such calibration.

In addition to controlling the load path through the sensor of the knee alignment device, it is critical that the test apparatus 200 do the same to obtain precision results. A loading plate may be applied on top of the sensor loading bars to accept forces anywhere on the loading plate. While the load to the sensor assembly 110 gets applied by two condyles during surgery, only one force is applied during calibration using force balance assumptions. For example, applying force in the middle should be equivalent to applying half the force equally on the right pressure sensor and the left pressure sensor.

The sensor assembly itself may be positioned using two pins that go through the two mounting holes shown in FIG. 13. A spring-loaded pin may push the sensor to take up any clearance between the hole in the pin. That mechanical assembly is part of a kinematic mount 200, so that different size sensors can be placed and replaced in a very consistent manner.

Broadly speaking, a kinematic mount is a precision mechanical system designed to precisely position and support an object, often used in optical and scientific instruments. It employs a set of precisely shaped and arranged components, such as spheres, pins, or wedges, which allow for controlled and stable movement in a specific direction while constraining motion in other unwanted degrees of freedom. Kinematic mounts are crucial in applications where precise alignment and stability are essential, such as in laser systems, telescopes, and microscopy setups. By providing controlled motion while minimizing vibrations and unwanted shifts, kinematic mounts ensure accurate and repeatable positioning, enabling the optimal performance of sensitive equipment in scientific and industrial settings.

Another key feature for improving the performance device is to focus on the nominal desired case where the forces are balanced between all the elements. The accuracy of the load measures will be greatest when the load is applied directly over the sensing element. However, it is desired to have an increased accuracy when the loads are applied equally among all four elements. This is done by developing an algorithm that weighs the accuracy of the desired balanced position to be higher than the outline cases because the sensing elements are located on the four corners of the sensor assembly.

During the calibration process, forces are applied at various locations 230 with the aid of a loading plate that may be positioned on top of the sensor assembly 110, see FIG. 14. Forces in the middle, in between, and directly over the sensing elements are applied at various force levels to calibrate the force and force centroid position.

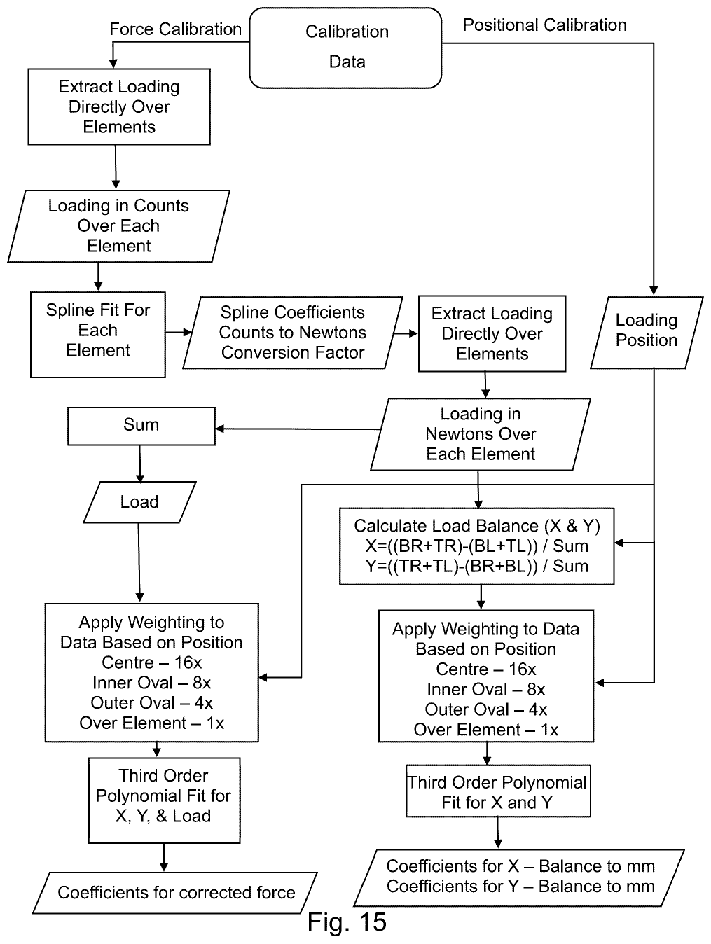

FIG. 15 presents an exemplary algorithm that may be used during the calibration process in order to process various locations and levels of forces applied using the loading plate in different positions of the sensor assembly 110. Since the objective of the knee replacement surgery is to have a balanced loading between the left and the right condyle and to know the total force applied during this condition, it is more important to know the force accurately when the load is balanced. Therefore, the calibration algorithm weighs the Force that's being applied at the center more heavily than when forces are being applied over the sensing elements, i.e. the load is not balanced. Forces applied outside the sensing element locations are considered invalid.

As shown in FIG. 15, the calibration for the system involves creating three key components:
1. Per-element calibration splines for converting raw counts from each element into force,
2. A polynomial fit for X & Y to convert load balance calculations into mm offsets from the center of the device, and
3. A polynomial fit that corrects the summed force from each of the four elements based on the position calculated by the position of force applied onto the sensor.

To calculate the parameters for the calibration, a calibration routine may be applied to the sensor within a controlled apparatus capable of providing known forces at known locations to the device under testing, as shown in FIGS. 13 and 14. At each loading point of the calibration, the position and force of the applied load are recorded along with the output of the sensor. To calculate the conversion from raw counts (uncalibrated sensor output) to force for each of the elements, the loading taken directly above each of the four elements is taken from the full set of data, such that there exist four subsets of calibration data consisting of applied force and sensor outputs for each of the elements when loaded.

Initially, this data is checked to verify that all four sensors are providing an output in raw counts under the same load to within X % of each other and not outside a nominal range of X+/−Y counts. This initial screening ensures that all four elements are operating correctly and within their expected tolerance.

Once the loading data over the elements is validated, a spline is then fitted, giving a means of conversion for each of the raw output counts to force. Here, the calibration can be calculated to determine the load position. This is done by means of calculating the load balance and applying a polynomial fit to refine the accuracy and convert it into mm space.

The load balance is calculated by:

$$\begin{matrix}a . & \\ {X}_{balance} = ( BottomRight + TopRight ) - ( BottomLeft + TopLeft ) BottomRight + TopRight + BottomLeft + TopLeft & \end{matrix}$$

$$\begin{matrix}b . & \\ {Y}_{balance} = ( TopLeft + TopRight ) - ( BottomLeft + BottomRight ) & \end{matrix}$$

BottomRight+TopRight+BottomLeft+TopLeft

To emphasize the fit of the polynomial around the center of the device, where accuracy is paramount, the data is weighted based on the location; it may also be grouped into four spatial regions corresponding to the corners of the sensors.

The center of the device, an inner oval, an outer oval, & the loading directly over the elements may be used to further adjust the data. In one example, based on the location, the data is replicated prior to being fitted by the following weightings:

$$\begin{matrix}a . & \\ Centre - 16 x & \end{matrix}$$

$$\begin{matrix}b . & \\ Inner Oval - 8 x & \end{matrix}$$

$$\begin{matrix}c . & \\ Outer Oval - 4 x & \end{matrix}$$

$$\begin{matrix}d . & \\ Over Elements - 1 x & \end{matrix}$$

Two polynomials are then fitted, one for the Y-axis using the Y-location of the applied in mm as the value being fitted to and the weighted Y_{balance} data as in input. Likewise, a similar procedure may be conducted for the X-axis using the X_{balance} and X load locations.

Finally, to refine the force value, a polynomial is fitted using the same weightings as with the load locations however, the independent variables being inputted to the fitting are the X-location and Y-location of the load in mm, and the summed total of the calibrated output of the four elements. The resulting output is a polynomial equation that corrects for force based on the load location.

The spline parameters for converting from counts to force, the X & Y position polynomials, & force refinement polynomial coefficients may then be sent to the device's onboard memory, the device reset, and a set of loadings at various positions may then be performed to verify the output of the device.

This portion of the specification describes novel tools designed to check the stiffness of the knee joint. Being able to do so during surgery is critical to patient satisfaction because a joint that is too loose causes instability, and a joint that is too tight causes stiff joints. Both can cause pain, and, if the tension on the left versus right condyle is not symmetrical, it can cause uneven and premature wear of the joint.

Whether a total knee replacement surgery is performed by traditional methods or with robotic assistance, the accuracy of the knee joint alignment depends on the cut and the thickness of the shim. The Precision Knee Aligner 100 described above is designed to provide immediate feedback to the surgeon on the compressive force between the knee joint as well as the balance between the left and right condyle. However, the surgeon cannot control the leg motion precisely to determine the axis and lateral stiffness of the joint. Literature research shows that the axis stiffness of the knee joint should be 100-1000 N/mm, and angular stiffness should be 0.1-1.5 Nm/deg.

A novel exoskeleton-type stiffness measuring mechanism 300 is proposed by the present invention that can be strapped to the patient's leg, using a Velcro strap or another suitable attachment method. It can apply a controlled amount of deflection to measure the stiffness of the knee joint, while the Precision Knee Aligner 100 provides the force measurement in between the knee joint.

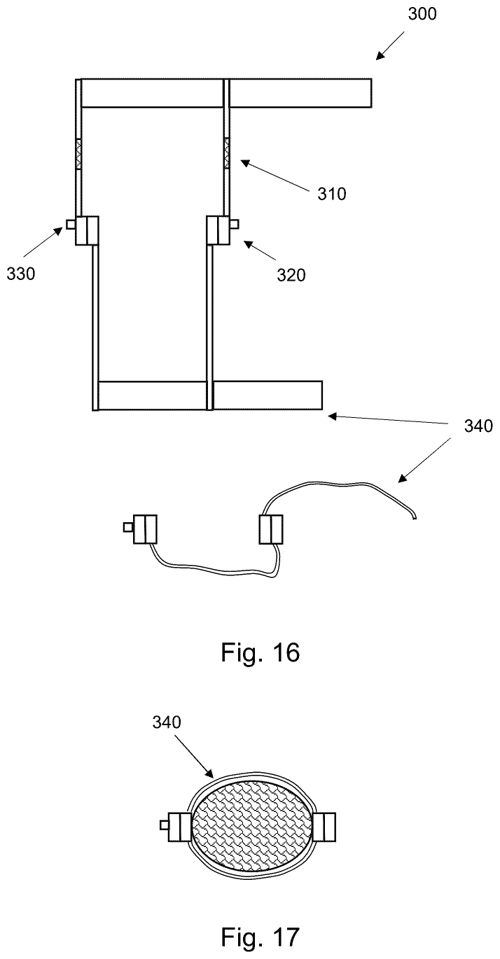

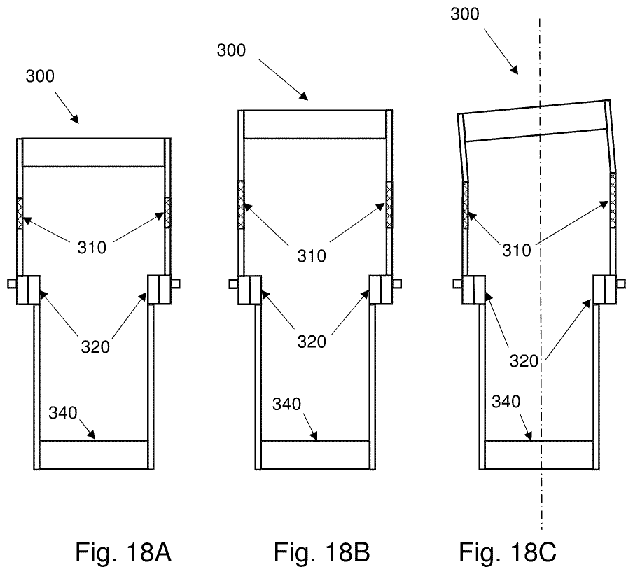

Measuring the medical lateral stiffness of the joint by rotating the joint perpendicular to the range of motion while measuring the angle can provide insight into the stiffness of the knee system. FIGS. 16, 17 and 18A through 18C show the device 300 components, including a pair of parallel linear actuators 310, a pair of rotating joints 320, at least one of them is equipped with a locking button 330, and an attachment fastener 340, such as a Velcro strap shown in use around the bone of the knee in FIG. 16. A rotating joint 320 that can be locked and linear actuators 310 on each side can make positional displacements to measure the force using the knee alignment device 100. Device 300 can be used to apply a controlled displacement to measure knee joint axial and angular stiffness in a consistent and repeatable manner after being applied to the patient. Various positions are shown in FIGS. 18, including a normal position in FIG. 18A, a linearly extended position in FIG. 18B, and a tilted position in FIG. 18C.

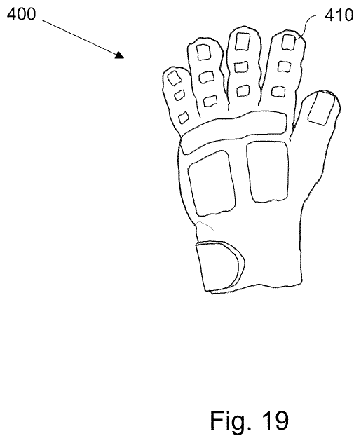

A further useful tool that can provide objective force measurements is seen schematically in FIG. 19. While an exoskeleton system 300 that straps onto the patient's knee and leg could provide more accurate measurements of the angle and stiffness of the knee joint, it may be is a more cumbersome procedure for the surgeon to deal with. Another method of measuring the stiffness while mimicking what a surgeon currently does is to instrument the surgeon's hand with a force-sensing glove 400 equipped with a plurality of force-sensing components 410. Since the force the surgeon needs to measure is just one value, the number of sensing elements 410 is not critical, and the glove design can be simplified to measure the force on the palm only with a single sensor 410. Using a built-in inertial measurement unit may be used to measure displacement while the surgeon applies a force to the knee joint. In that case, the glove 400 may be configured to measure both force and displacement, which allows an automated calculation of the knee joint stiffness.

The following portion of the specification describes the issues surrounding knee alignment steps taken during surgery and the tools proposed to assist in bone cutting during these steps as part of the present invention.

Precise alignment of the knee prosthesis is a central factor that determines the success of this procedure and plays a substantial role in ensuring the longevity of the implant, refining knee biomechanics, and ameliorating postoperative functions.

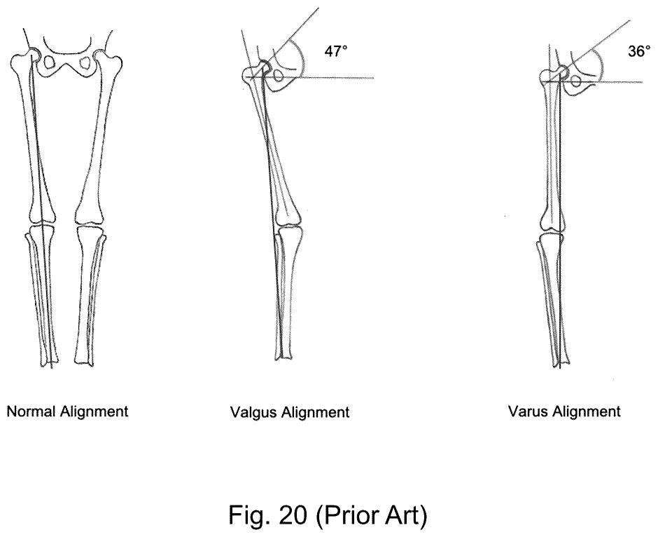

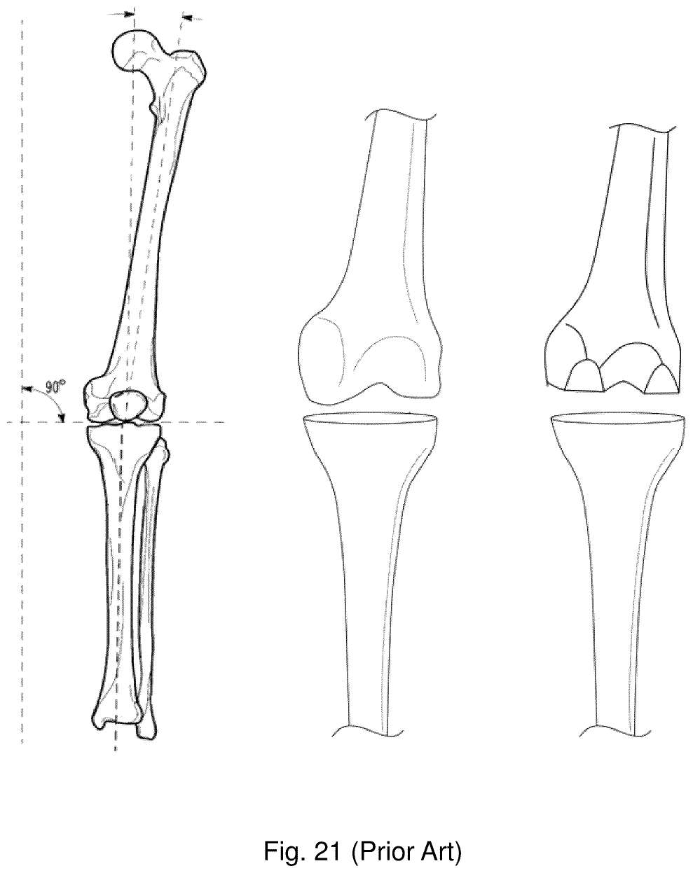

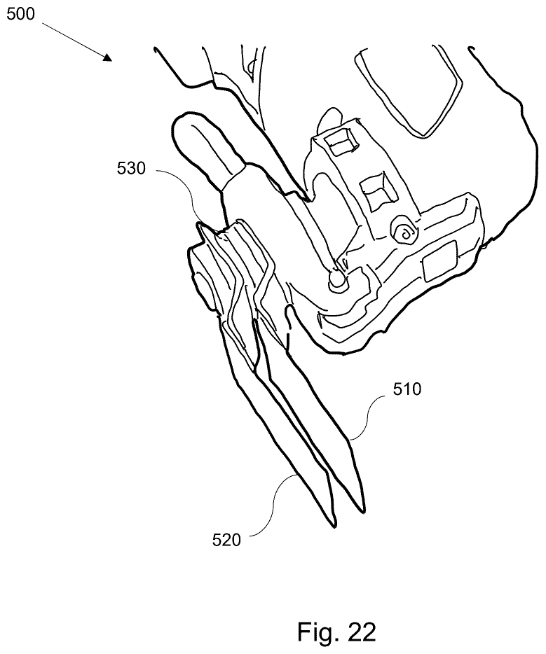

Traditionally, the orthopedic community has practiced various alignment philosophies. One conventional method is mechanical alignment. This technique seeks to align the prosthesis components to manifest a neutral mechanical axis of the limb, which is approximately 0°±3° varus/valgus, see FIG. 20. While this method is historically accepted and boasts long-term success in numerous cases due to its predictability, it might not be ideal for every patient. Some reports suggest a heightened risk of aseptic loosening or early wear because the alignment might not mirror the patient's most “natural” stance.

Emerging from the traditional method, the anatomical or kinematic alignment has gained traction. This alignment approach aims to emulate the patient's native or pre-disease knee alignment, which may incline slightly towards the varus or the valgus rather than be a strict mechanical neutral. By respecting individual anatomy, there's a potential promise of offering postoperative kinematics that feels more intuitive and natural to the patient. However, it is less rooted in historical practice and necessitates meticulous preoperative planning. The longevity and wear of this technique remain subjects of continued research.

Another progressive technique is soft tissue alignment, which underscores the equilibrium of soft tissues, including ligaments and tendons, during the positioning of prosthetic components. The allure of this method is the potential to yield a balanced knee, possibly leading to enhanced postoperative functions and patient contentment, complemented by a more organic range of motion. Yet, it's worth noting the technical demands of this approach, requiring seasoned expertise for consistency and risking potential over-tightening or over-releasing of structures.

In the orthopedic arena, opinions have evolved over time concerning the optimal alignment strategy for total knee arthroplasty. Although mechanical alignment has remained a benchmark for years, contemporary research and clinical experiences are advocating the merits of anatomical and soft tissue alignments. Many surgeons now champion a customized approach, adapting the alignment strategy to a patient's unique anatomy and requirements. However, the overarching consensus on a universally “best” alignment technique remains elusive, necessitating continued research and review of emerging evidence-based practices for a holistic understanding.

Whether the desired alignment is mechanical, anatomical, or kinematic (see left panel in FIG. 21), the cut between the bones should be parallel. Making a first cut (middle panel in FIG. 21) and then making the second cut (right panel in FIG. 21) may be challenging when they are done one after another, especially since the actual knee joint anatomy is not rectilinear.

A dual-blade cutting device 500 may assist in making a precision cut during knee replacement surgery, as seen in FIG. 22. The saw 500 may include a first blade 510 separated from the parallel blade 520 by a spacer 530, which may be selected to have any desired thickness so as to define the distance between the cuts. To use this saw 500, the surgeon aligns the leg to the desired alignment, secures it, and makes the cut to both bones at the same time, thus achieving a parallel cut in a single operation that maintains the desired alignment between the bones. The dual-blade cutting device may be configured to have the parallel blades moving synchronously and both moving in the same direction. In other embodiments, the parallel blades may be configured to move in opposite directions so as to compensate for any inertial forces, which may improve the accuracy and reduce the handling forces required to maintain the device in the desired position.

### Knee Alignment Measurement Instrument

Long-term success and implant survivorship following total knee arthroplasty depend on two critical mechanical factors: accurate implant alignment and consistent soft-tissue balance throughout the entire range of motion. While robotic and computer-assisted systems have substantially improved the precision and reproducibility of bone resections and component positioning, the assessment of soft-tissue forces and the optimization of joint gap spacing remain largely manual and subjective processes. Improper soft-tissue balance is a known cause of postoperative instability and represents a major contributor to patient dissatisfaction after joint replacement, accounting for a significant portion of revision procedures. The potential consequences of poor soft tissue balancing also include abnormal gait kinematics, elevated contact stress within the joint leading to early failure, patellofemoral mal-tracking with anterior knee pain, and aseptic loosening. Achieving the correct balance between the femur and tibia components requires both accurate force measurement and precise control of the distance between these structures during the trialing phase of surgery.

Conventional knee replacement techniques currently rely heavily on manual feedback, spacer blocks, and mechanical tensor devices, all of which are subject to observer-dependent variability. The absence of quantitative intraoperative data prevents objective measurement of compartmental loads and limits the surgeon's ability to optimize joint mechanics, contributing to substantial intra-surgeon variability. A critical component of this balancing process involves selecting the appropriate tibial insert thickness from a range of interchangeable shims of various thicknesses, each defining a specific distance between the femur-facing surface and the knee alignment measurement tool. Under-stuffing the joint with an insufficiently thick shim results in laxity, unstable kinematics, and reduced forces throughout flexion, while over-stuffing with an excessively thick shim produces elevated compartmental loads, restricted rollback, and diminished functional flexion range. Current practice requires the surgeon to iteratively insert and remove rigid shims during trialing, relying on surgeon's tactile feedback to assess stability at each thickness. This process is both time-consuming and inherently subjective, making it difficult to identify the optimal stack height that balances force distribution and preserves physiological joint mechanics.

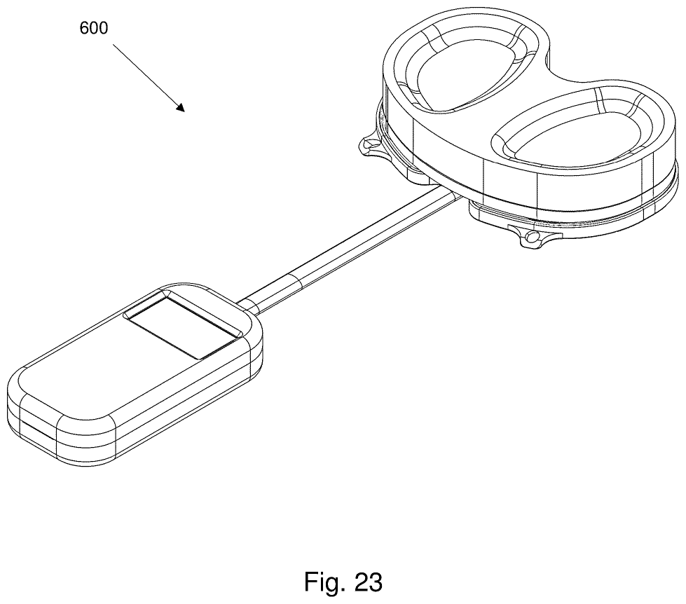

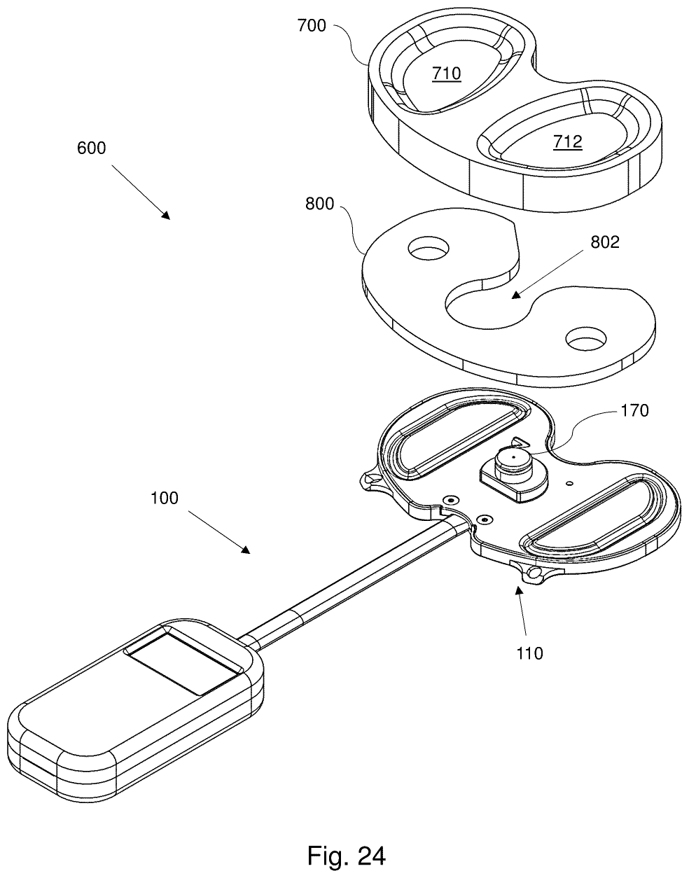

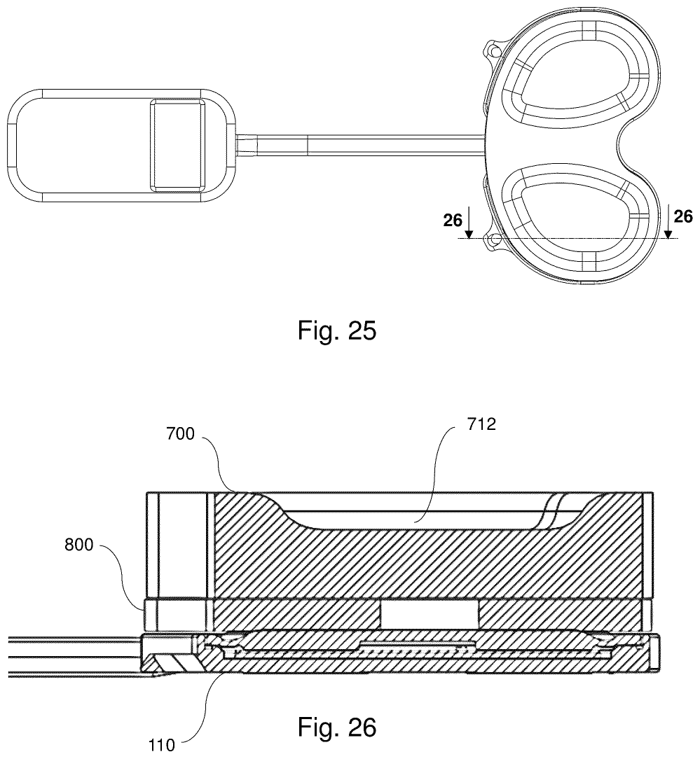

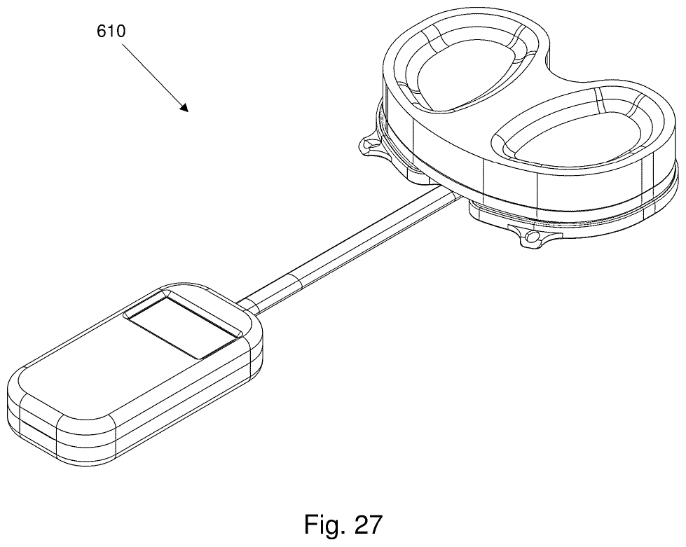

To address these limitations, the present invention describes a knee alignment measurement instrument 600, such as shown in FIGS. 23-26, which is a kit comprising a femur-facing insert 700, a knee alignment measurement tool 100 positioned, in one example, therebelow, and one or more shims 800 configured to be inserted between the femur-facing insert 700 and the knee alignment measurement tool 100. Such an instrument is expected to provide substantial clinical advantages. The femur-facing insert 700 comprises a femoral contacting surface configured, with two depressions 710 and 712, to face the femur and to stay in contact therewith during the knee alignment measurement procedure, thereby transmitting compressive forces from the femoral component through the stack to the tibial side. The knee alignment measurement tool 100 is described above in greater detail. It includes a sensor assembly 110 with a rigid base supporting a left sensor and a right sensor positioned adjacent to one another, and a cover configured to transmit compression force applied on top thereof individually to the left sensor and the right sensor, enabling real-time feedback on both total joint force and the balance of forces between the left condyle and the right condyle. A display unit operatively connected to the sensor assembly powers up and operatively controls the left sensor and the right sensor and displays the readings therefrom to provide the surgeon with an objective, quantitative assessment of compartmental loading throughout the flexion arc. By stacking these components and varying the shim thickness, the surgeon can systematically adjust the total height of the stack and directly observe the corresponding changes in force magnitude, force distribution, and medial-lateral balance. This replaces subjective tactile assessment with objective data that enables more reproducible soft-tissue adjustments and optimal implant selection.

The knee alignment measurement instrument may be configured so that the sensor assembly 110, the shim 800, and the femur-facing insert 700 together form a compact, modular stack that can be readily inserted into and removed from the joint space during total knee arthroplasty. The sensor assembly 110 is positioned at the bottom of the stack and is configured to rest against the tibial side, for example, on the resected tibial surface. The shim 800 is then placed on top of the sensor assembly 110, and the femur-facing insert 700 is positioned on top of the shim 800 so that its femoral surface faces and contacts the femur (trial) implant. In this arrangement, compressive forces generated between the femur and the tibia during trialing are transmitted through the femur-facing insert 700, through the shim 800, into the sensor assembly 110, and then to the underlying structures. The center post 170 extending from the sensor assembly 110 and the corresponding central opening 802 in the shim 800 cooperate to keep the shim 800 properly centered and aligned, while pins or other positioning elements of the femur-facing insert 700 further ensure that all three components remain coaxial and stable under load.

A plurality of planar shims 800 may be supplied to the surgeon, each having the same external profile and central opening 802 but differing in thickness. The thickness of the shims may range from about 1 mm to about 15 mm, with increments of 1 mm or 2 mm between adjacent shim sizes, which allows for fine adjustment of the total height of the stack. Each shim 800 slides over the center post 170 using the central opening 802, which is sized and shaped to facilitate quick placement without excessive play, thereby maintaining accurate alignment relative to the sensor assembly 110. The femur-facing insert 700 may include mating holes, slots, or protruding pins that interface with corresponding features on the shim 800 and/or the sensor assembly 110 so that the components do not shift laterally during insertion, removal, or loading. This configuration allows the shim 800 to change the distance between the femur-facing insert 700 and the knee alignment measurement tool while preserving the desired relationship between the left sensor, the right sensor, and the femoral condyles.

During surgery, the surgeon may select an initial shim 800 of a nominal thickness, place it over the center post 170 on the sensor assembly 110, and then position the femur-facing insert 700 on top of the shim. The assembled stack is then inserted between the femur and the tibia so that the femur-facing insert 700 contacts the femoral component and the sensor assembly 110 rests on the tibial side. The surgeon may cycle the knee through extension and flexion while observing the readings from the knee alignment measurement tool, which provide quantitative information regarding total compressive force and medial-lateral balance. If the surgeon determines that the joint is understuffed, for example, if forces are low and the joint feels lax, the stack may be removed, the current shim 800 lifted off the center post 170, and a thicker shim 800 placed in its stead. Conversely, if the forces are excessively high or motion is restricted, a thinner shim may be substituted. This trial procedure may be repeated as needed, with different shims being placed over the center post 170, until an appropriate combination of force, alignment, and stability is achieved throughout the range of motion.

Once a satisfactory shim thickness is identified using the femur-facing insert 700, the shim 800, and the sensor assembly 110, the corresponding joint gap and loading condition can inform the selection of the final tibial insert or implant configuration. Because each shim 800 is precisely manufactured and referenced to the center post 170 and alignment features of the femur-facing insert 700, the surgeon can correlate a chosen shim thickness with a desired insert thickness or target gap. The use of the knee alignment measurement tool in conjunction with the stack of interchangeable shims 800, therefore, provides an efficient workflow in which the surgeon can quickly iterate through different stack heights while relying on objective force data and consistent component positioning, improving the likelihood of achieving balanced soft tissues and proper implant alignment in the completed knee replacement

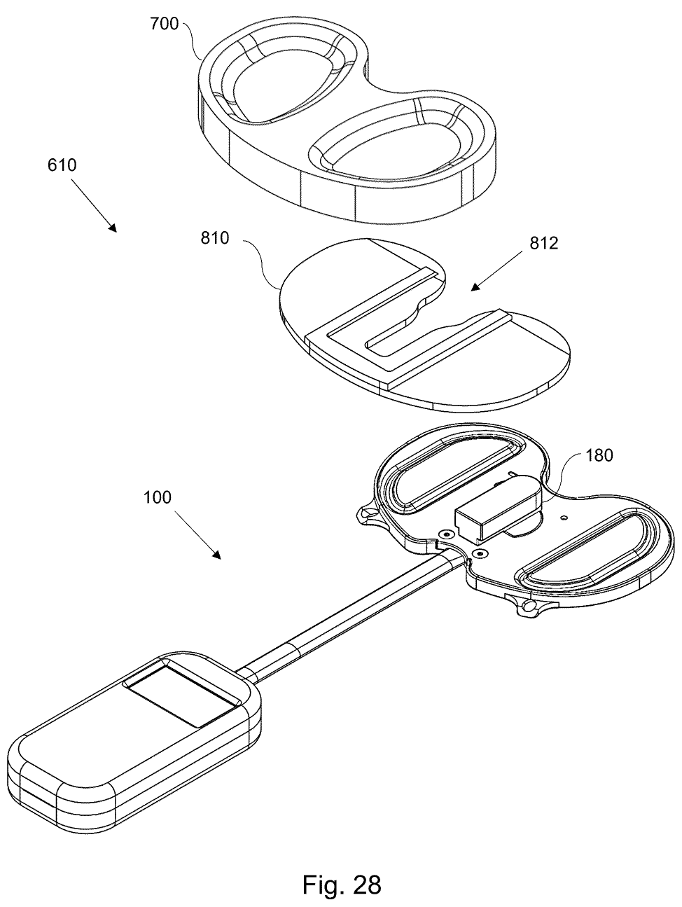

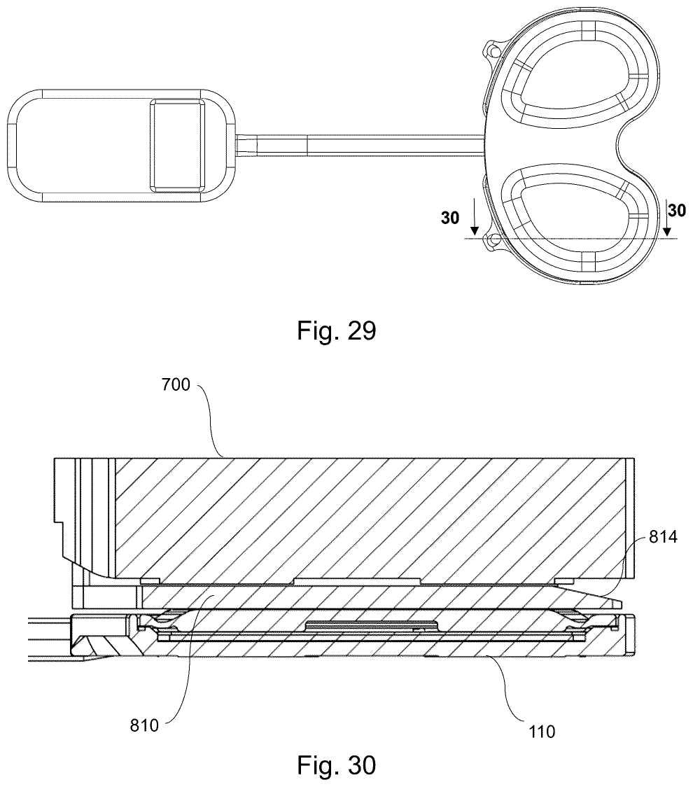

An alternative configuration of the knee alignment measuring tool 610 is seen in FIGS. 27-30, in which the basic stack architecture is preserved, but the way the shim is attached and exchanged is modified. In this embodiment, the sensor assembly and femur-facing insert remain generally similar in function, but the central locating structure is changed with the center post 170 made in the form of a slide post 180. The shim 810 is no longer constrained by a circular central opening, but instead includes an elongated opening 812 that is shaped to receive the slide post 180 from the side. This design maintains stable alignment of the shim 810 relative to the sensor assembly and femur-facing insert while creating a lateral entry path that allows the shim to be inserted and removed without disassembling the rest of the stack.

The elongated opening 812 of the shim 810 may be dimensioned so that one end of the opening forms a seat or pocket that surrounds the slide post 180 when the shim is fully engaged. The other end of the opening may extend toward the periphery of the shim to create a slot or channel that allows the shim to be moved laterally on or off the slide post 180. The slide post 180 may, in turn, include features such as a narrowed neck, a chamfered lead-in, or a slight undercut that guide the shim 810 into its seated position and resist accidental disengagement during loading. The femur-facing insert in this configuration may include pins or other positioning elements that press the shim 810 flat against the sensor assembly and work together with the slide post 180 to prevent rotation and lateral shift while the knee is cycled through flexion and extension.

In use, the surgeon may begin by placing an initial shim 810 onto the slide post 180 by sliding the elongated opening 812 along the axis of the opening until the shim is fully seated around the slide post. The femur-facing insert 700 is then positioned on top of the shim, and the assembled stack is inserted between the femur and the tibia. The surgeon can assess total force, medial-lateral balance, and joint stability with the knee alignment measuring tool 610 in place. If the measured forces and joint feel indicate that a different stack height is needed, the surgeon does not need to remove the entire stack from the joint. Instead, the load can be partially released, the femur-facing insert slightly lifted or rocked to unload the shim, and the shim 810 can then be slid out along the elongated opening 812 away from the slide post 180. A replacement shim 810 with a different thickness can be slid into engagement with the slide post 180 and repositioned under the femur-facing insert while the sensor assembly remains in situ on the tibial side.

This alternative configuration of the knee alignment measuring tool 610 therefore improves surgical workflow by reducing the number of insertion and removal cycles required to identify the optimal shim thickness. The ability to exchange shims 810 laterally along the slide post 180 while leaving the sensor assembly 110 and femur-facing insert 700 substantially in place can shorten trialing time, reduce manipulation of soft tissues, and help preserve the consistency of component alignment from one trial condition to the next. Because the same sensor assembly 110 remains seated on the tibial side throughout the process, the force readings from the left and right sensors remain directly comparable across different shim thicknesses, supporting more precise and reproducible selection of the final implant configuration.

### Inflatable Shim

The present invention further describes an inflatable shim 850 configured to gradually adjust the distance between the femur and the tibia during the knee alignment measurement procedure, thereby complementing and improving on the discrete, thickness-based adjustments provided by planar shims. In contrast to prior embodiments in which the joint gap can only be changed in fixed increments by exchanging one rigid shim for another, the inflatable shim 850 allows the distance to be adjusted to essentially any desired value within the range of inflation of the inflatable bladder 860. This enables the surgeon to fine-tune the joint space in small, controlled steps while monitoring the corresponding changes in force and balance on the knee alignment measurement tool, improving the precision with which an optimal alignment and soft-tissue balance can be identified.

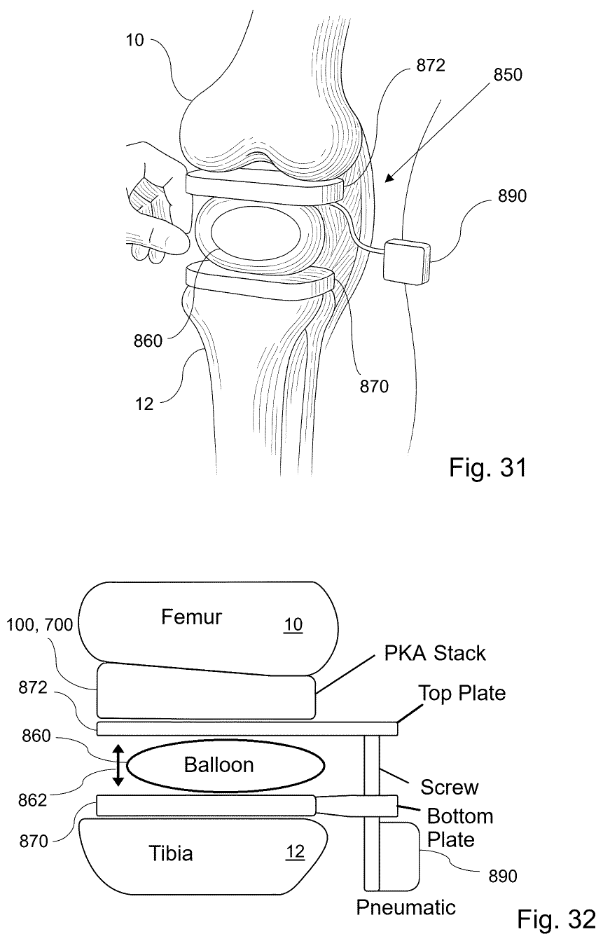

The concept of the inflatable shim 850 is illustrated in FIGS. 31 and 32. The inflatable shim 850 may include an inflatable bladder 860 sandwiched between at least one rigid shim base 870 and, in some embodiments, a second rigid plate 872 located on the opposite side of the inflatable bladder 860. The rigid bases 870, 872 serve to distribute load over the surface of the inflatable bladder 860 and to provide well-defined planar contact surfaces toward the tibial side and the knee alignment measurement tool 100. A distance sensor 862 is provided to measure the distance between the plates 870 and 872, which is indicative of the current thickness of the inflatable shim 850 and, therefore, of the total height of the stack of components. An inflation port may be connected to a manual or automated source of a suitable fluid 890, such as medical-grade air, which can be controlled by the surgeon or operated automatically to inflate or deflate the inflatable bladder 860. Gradual ingress or egress of the fluid causes the distance between the plates 870 and 872 to increase or decrease in a continuous manner rather than in a step-wise manner as in previous embodiments. The stack of components forming the knee alignment measurement instrument in this configuration, as shown in FIG. 32, includes the inflatable shim 850 positioned over the tibia 12, followed by the knee alignment measurement tool 100 and the femur-facing insert 700 positioned to be in contact with the femur 10.

The distance sensor 862 of the inflatable shim 850 may be a capacitive distance-measuring sensor or a lidar sensor configured to measure the distance using pulsed laser light, thereby providing continuous real-time feedback on the separation between the rigid plates 870 and 872. By coupling the output of the distance sensor 862 with the force measurements from the left and right sensors of the knee alignment measurement tool 100, the system can present the surgeon with synchronized information on joint gap height, total compressive force, and medial-lateral balance at each degree of inflation. In one example, the distance sensor 862 may be embedded within one of the rigid plates 870 or 872 and directed toward the opposite plate so that changes in separation are directly translated into changes in a measurable electrical or optical signal. In another example, the distance sensor 862 may be mounted off to the side of the inflatable bladder 860 while still spanning the distance between the tibial side and the knee alignment measurement tool 100. In either case, the measured distance may be processed and displayed by the same display unit that processes the force signals, allowing the surgeon to correlate specific joint gap values with desired force profiles and balance conditions.

The inflatable bladder 860 itself may be made from a biocompatible material selected to provide appropriate flexibility, durability, and resistance to puncture under intraoperative loading conditions. Suitable materials include silicone, nylon, polyethylene terephthalate (PET), polyethylene, polyurethane, Pebax, or blends of these or other compatible polymers. The film thickness and layup may be chosen to allow repeated inflation and deflation cycles without fatigue-related failure, while maintaining a smooth exterior surface that can be easily cleaned or covered by a sterile barrier as needed. The bladder 860 may be formed to have multiple layers of polymer, may have reinforced seams, or can be produced by heat sealing, radio-frequency welding, or adhesive bonding of two or more film layers so as to define an internal cavity that inflates uniformly when pressurized. In some embodiments, internal baffles or weld lines may be incorporated to control the shape of the bladder 860 when inflated, ensuring that the expansion remains primarily in the vertical direction and that the load is transmitted evenly to the plates 870 and 872 and, ultimately, to the knee alignment measurement tool 100 and femur-facing insert 700.

The rigid shim base may be made to be detachable from the inflatable bladder so as to facilitate reusing of the rigid component while discarding the inflatable bladder after every procedure. The inflatable shim 850 may be configured to be compatible with both cruciate-retaining and posterior-stabilized knee implant systems.

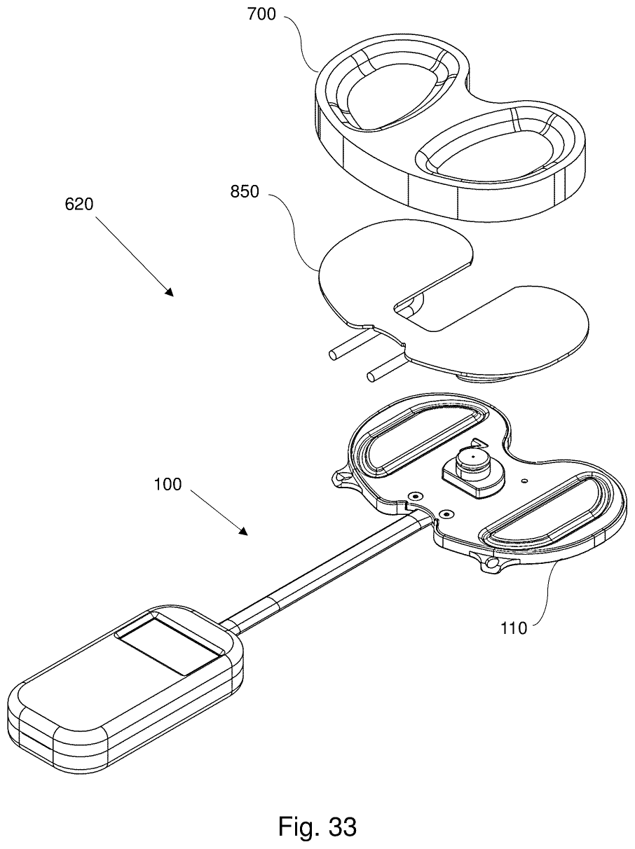

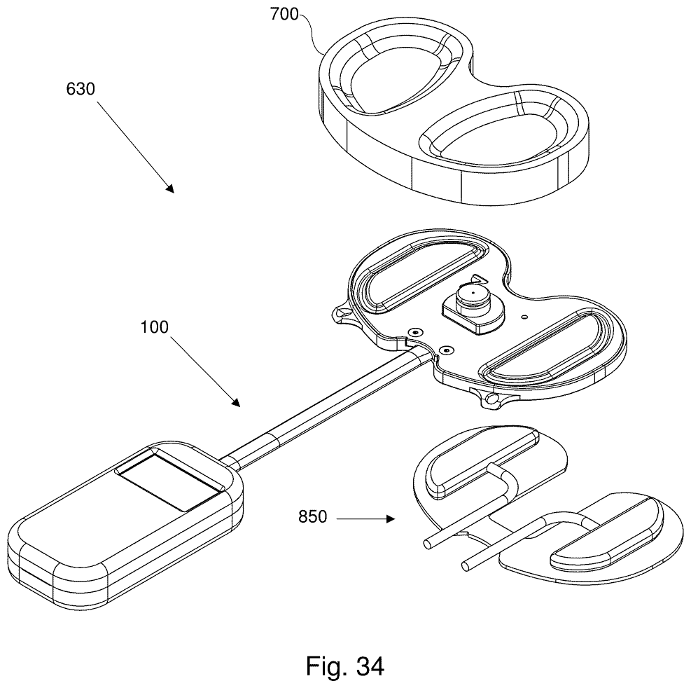

An embodiment of the knee alignment measurement tool 620 is shown in FIG. 33, where the inflatable shim 850 is positioned between the sensor assembly 110 and the femur-facing insert 700, with the inflatable bladder oriented toward the sensor assembly 110. In this arrangement, inflation alters the distance directly above the sensors, enabling the surgeon to observe immediate changes in total compressive force and medial-lateral balance as the stack height increases. Alignment features on the femur-facing insert 700 and the sensor assembly 110 may engage the rigid portions of the inflatable shim 850 to maintain coaxial positioning during insertion and inflation, reducing lateral shift as the bladder expands. In an alternative configuration illustrated in FIG. 34, the knee alignment measurement tool 630 places the inflatable shim 850 below the sensor assembly 110 so that expansion occurs against the tibial side. This placement can be advantageous when access or routing of the inflation line is more reliable near the tibial margin, and it preserves the contact interface between the femur-facing insert 700 and the sensor cover while still providing continuous height adjustment within the stack.

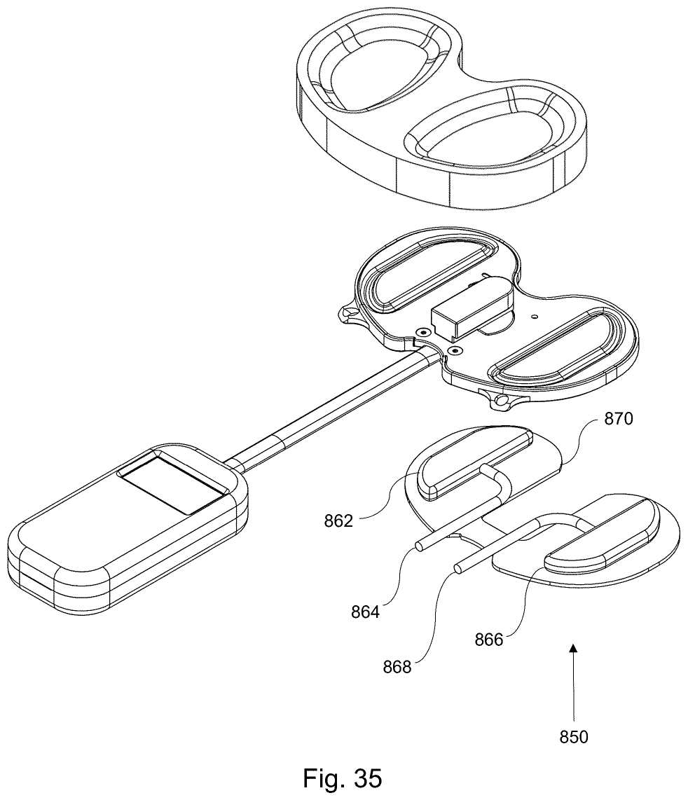

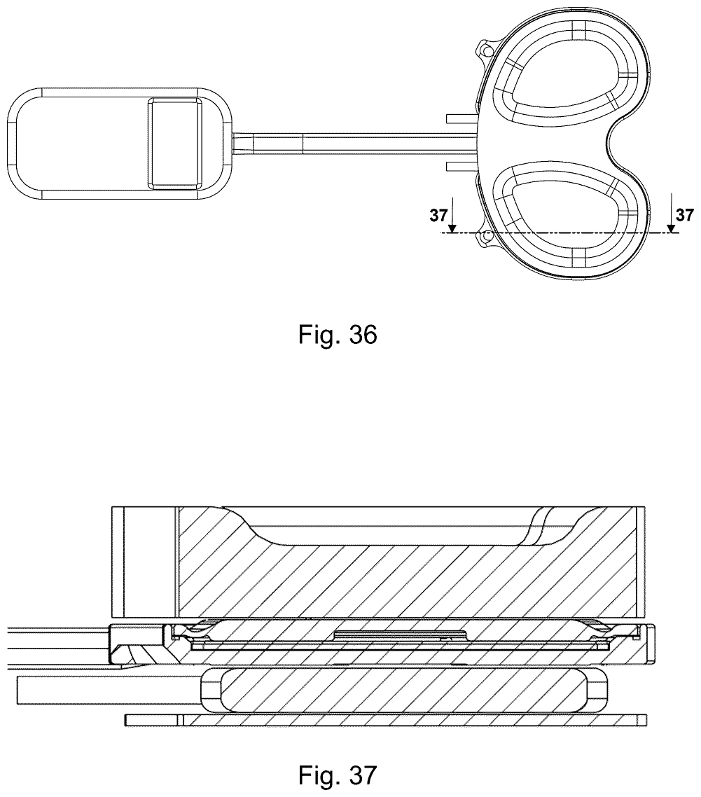

FIG. 35 illustrates a version that incorporates a slide post for lateral insertion and removal of the inflatable shim 850 from one side of the stack without disassembling the remaining components. In this configuration, the inflatable shim 850 includes a slot or elongated opening that engages with the slide post, allowing the shim to be slid into a seated position underneath the femur-facing insert 700 or, in the alternative layout, beneath the sensor assembly 110. This approach improves workflow by allowing the surgeon to exchange or service the inflatable shim in situ, such as replacing a bladder or switching to a different shim format, while keeping the sensor assembly 110 and the femur-facing insert 700 substantially undisturbed. FIGS. 36 and 37 present a top view and a cross-sectional view of these configurations, showing how the slide post, alignment pins, and shim seating surfaces cooperate to prevent rotation and migration during flexion-extension cycles.

Further details of the inflatable shim 850 are provided in FIG. 35. The shim comprises a rigid shim base 870 and an inflatable bladder that includes a pair of inflatable balloons 862 and 866, each with a corresponding inflation port 864 and 868. The two balloons can be actuated independently to allow compartment-specific adjustments or interconnected to inflate and deflate together for symmetric changes in gap height. Independent control can be used to bias loading toward the medial or lateral side to evaluate the effect of selective tensioning while monitoring the left and right sensor outputs in real time. When linked, a single inflation line simplifies setup and produces uniform height changes across the contact surface. The inflatable shim 850 may provide an adjustable height range from about 1 mm to about 8 mm above the fully deflated state, enabling fine, continuous tuning within clinically useful bounds during trialing.

In some alternative embodiments, the force measurement function of the knee alignment measurement instrument may be implemented without using a left sensor and a right sensor within the sensor assembly 110. Instead, the compressive forces between the femur 10 and the tibia 12 may be inferred from the air pressure inside the inflatable bladder 860 of the inflatable shim 850. In this configuration, the inflatable bladder 860 is in communication with at least one pressure transducer configured to measure the internal pressure of the bladder as it is compressed between the tibial side and the femur-facing cap 700. Because the geometry and contact area of the inflatable bladder 860 are known, changes in internal pressure may be calibrated to correspond to applied joint forces. The pressure transducer output may be routed to the display unit of the knee alignment measurement tool, which then displays real-time estimates of total joint force as the surgeon adjusts the degree of inflation and cycles the knee through flexion and extension.

In further alternative embodiments, the inflatable shim 850 includes a pair of inflatable balloons 862 and 866 corresponding to the medial and lateral compartments, each having its own inflation port 864, 868 and its own pressure sensor. In this case, the left balloon 862 is positioned under the medial condyle region and the right balloon 866 under the lateral condyle region, so that the pressure measured in each balloon reflects the load borne by the corresponding side of the joint. Independent pressure readings from the left and right balloons may be used to calculate both total compressive force and the balance of forces between the left compartment and the right compartment, without requiring separate solid-state force sensors within the sensor assembly. The balloons may be inflated from a common source of medical-grade air, with flow split through controllable valves, or from separate sources, allowing either symmetric inflation or controlled differential inflation while still providing compartment-specific pressure and force information.

In some versions, the pressure-based force measurement may be used alone, with the sensor assembly 110 simplified to a passive mechanical support, while in other versions, the pressure sensing may be combined with conventional left and right sensors to provide redundant or cross-validated measurements. In all of these embodiments, the pressure transducers may be located either directly on the inflatable shim 850, integrated into the inflation ports 864, 868, or positioned remotely along the inflation tubing, provided that the volume between the balloon and the transducer is small enough to maintain an accurate relationship between internal pressure and applied load. During use, a calibration procedure may be performed to map pressure values to known forces, after which the system can display real-time force and balance information derived solely from the air pressure in the inflatable bladder 860 or in the left balloon 862 and right balloon 866 while the joint gap is adjusted intraoperatively.

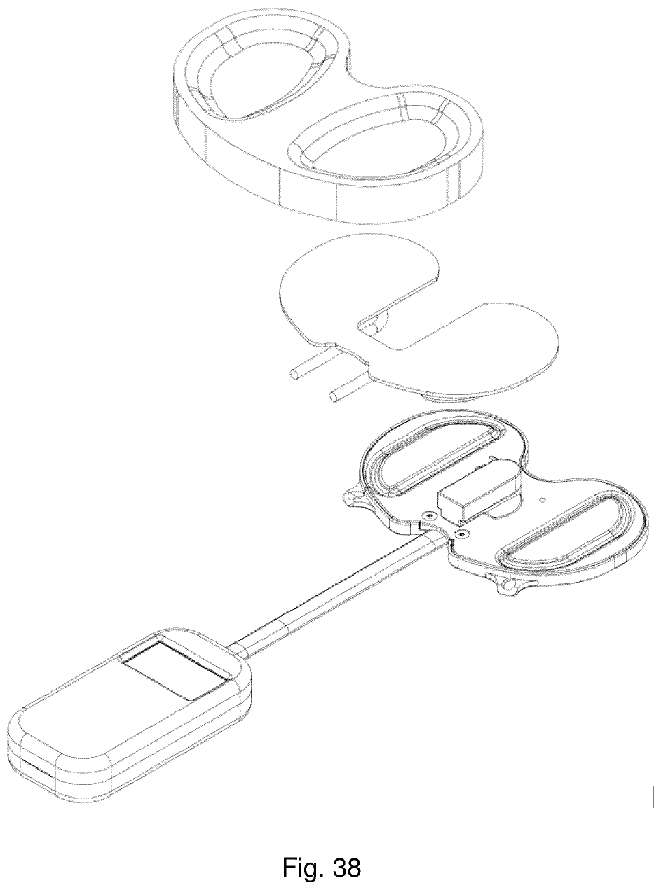

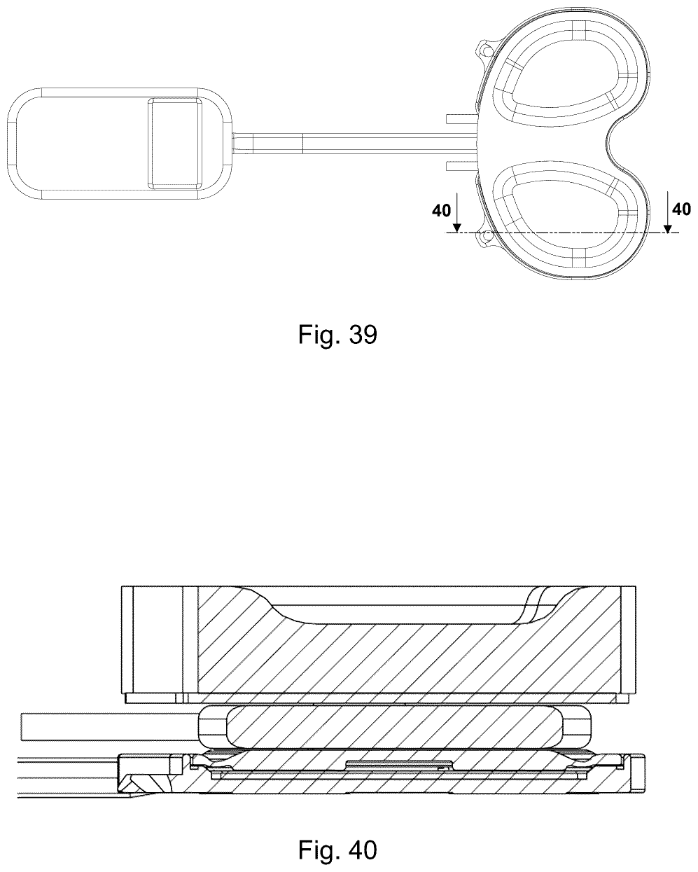

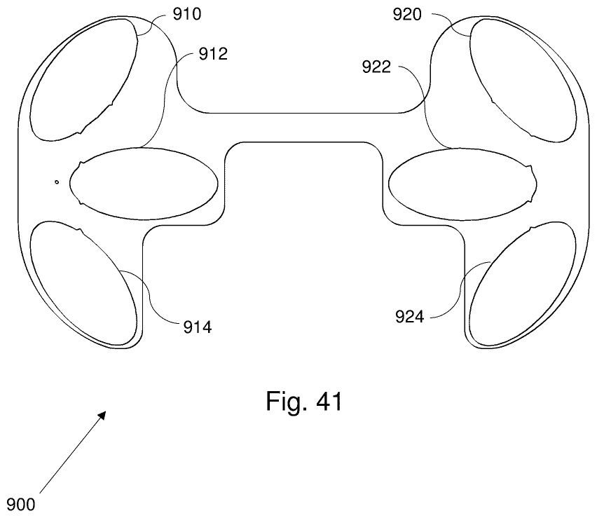

FIGS. 38-40 show a further embodiment in which the inflatable shim is positioned between the sensor assembly and the femur-facing insert.

In use, the surgeon positions the inflatable shim 850 in the desired location within the stack of components forming the knee alignment measurement instrument, such as between the tibia 12 and the sensor assembly 110 or between the sensor assembly 110 and the femur-facing insert 700, and routes the inflation port or ports to a sterile, calibrated pump system. The device is used intraoperatively following standard bone cuts and initial trial component placement. With the inflatable bladder 860 fully deflated, the inflatable shim 850 is inserted so that the rigid shim base 870 and any upper plate 872 are correctly aligned with the joint line and do not impinge on surrounding soft tissues. The inflation port or ports 864, 868 are then connected to a source of medical-grade air that complies with ISO 7396-1, and an airtight seal and zero baseline pressure are verified before inflation begins.

During the trialing procedure, the surgeon incrementally inflates one or both balloons 862, 866 while observing the corresponding changes in joint opening and load distribution. Gradual ingress of air increases the height of the inflatable shim 850 from its fully deflated state by up to about 1 mm to about 8 mm, producing proportional changes in the separation between the femur 10 and tibia 12. The distance sensor 862 of the inflatable shim provides continuous real-time feedback on the separation between the rigid plates 870 and 872, while the knee alignment measurement tool 100 simultaneously measures total compressive force and the balance of forces between the left condyle and the right condyle. Under valgus and varus stress, the surgeon can evaluate medial and lateral joint space symmetry, record distraction values, and correlate these measurements with data from navigation systems or from a smart tibial force sensor integrated into the trial components if available.

Based on the observed asymmetries in joint gap and condylar loading, the surgeon may perform targeted soft tissue releases or adjust component positioning and then repeat the inflation and assessment cycle until the desired combination of gap height, force magnitude, and medial-lateral balance is achieved. The inflatable shim 850 may be used together with digital tension sensors, robotic guidance systems, or manual measurement tools, and is configured so as not to interfere with standard instrumentation or surgical workflow. Once a satisfactory condition is confirmed and the appropriate equivalent thickness for the final construct has been determined, the inflatable bladder 860 is fully deflated, the inflatable shim 850 is removed from the stack, and the final tibial insert and femoral components are implanted. The shim may then be inspected for integrity, and the joint space is checked to ensure that no residual material remains before closure.

The inflatable shim 850 of the present invention offers several advantages compared to traditional soft tissue balancing techniques used during knee replacement surgery. It provides objective, quantifiable information about joint space distraction by combining the adjustable inflatable bladder 860 with the integrated distance sensor 862, rather than relying solely on the surgeon's tactile feel. This objective measurement of the separation between the femur 10 and tibia 12, together with the force readings from the knee alignment measurement tool 100, reduces dependence on subjective assessment and helps the surgeon identify a more precise and reproducible balance condition.

Because the inflatable shim 850 can be used with a standardized inflation protocol, it enables more consistent evaluation across different cases and operators. Incremental changes in inflation volume can be correlated with specific changes in joint gap height and condylar loading, making it easier to reproduce a preferred balancing strategy from one procedure to another. This capability supports both mechanically aligned and kinematically aligned workflows, allowing the surgeon to tailor the joint gap and force distribution intraoperatively based on patient-specific anatomy rather than a fixed set of spacer thicknesses.

The inflatable shim 850 also minimizes the risk of over-stuffing the joint by allowing the surgeon to observe force and gap behavior while progressively increasing joint distraction. Rather than committing to a fixed insert thickness based on initial assessment, the surgeon can incrementally inflate the inflatable bladder 860 and directly observe how changes in joint gap height affect total compressive force and medial-lateral balance on the knee alignment measurement tool 100. This staged, data-driven approach enables the surgeon to identify the optimal gap height that achieves balanced loading without exceeding desired force targets. By avoiding unnecessary over-stuffing, which can restrict motion and create excessive contact stresses, the inflatable shim 850 helps preserve normal knee kinematics and reduces the risk of postoperative complications related to tight, over-constrained implants. The inflatable shim 850 is designed to be low profile and compatible with standard trial components, so it introduces minimal disruption to native joint mechanics during assessment and can be readily integrated into existing intraoperative workflows.

Clinical work with objective balancing tools has demonstrated that quantitative methods for assessing joint space and compartmental loading can reduce variability and are associated with improved functional outcomes after total knee arthroplasty. By providing a simple, scalable means to generate real-time measurements of joint gap and tibiofemoral forces without significantly altering surgical technique, the inflatable bladder shim 850 supports this trend toward data-driven balancing and offers a practical path to more reliable and personalized soft tissue management in the operating room.

Improved Accuracy with Thermal Compensation

FIG. 41 shows a sensor configuration that goes beyond just a single left sensor and a single right sensor, as was described for previous embodiments. In this configuration, the left side of the knee alignment measurement tool includes a group of left sensors 910, 912, and 914, while the right side includes a group of right sensors 920, 922, and 924. These multiple elements may be arranged in some embodiments so that at least one sensor on each side is designated as a primary load-bearing sensor, with the remaining sensors configured to either share the load or serve as reference elements. In the embodiment shown in FIG. 41, two sensors 910 and 914 of the group of left sensors and two sensors 920 and 924 of the right group of sensors are load-bearing sensors. One sensor from each of the group of left sensors and the group of right sensors may be selected to be non-load-bearing, for example, sensors 912 and 922. To form a non-load-bearing sensor, the cover of the sensor assembly may be configured not to have a thicker portion arranged above the sensor, so that no significant compressive load is transmitted to that sensor during use, while the surrounding structure remains otherwise similar to the load-bearing sensors.

The purpose of at least one or more of these non-load-bearing sensors is to provide an in situ reference signal that reflects thermal expansion and other environmental effects that act on the sensor assembly as a whole, rather than mechanical loading from the femoral condyles. When the knee alignment measurement tool is moved from a room-temperature environment into the operating field, the sensor assembly may warm toward body temperature or experience other ambient changes that can alter the electrical response of the sensing elements. Since the non-load-bearing sensors 912 and 922 are exposed to the same temperature and environmental conditions as the load-bearing sensors, but are mechanically shielded from compressive forces, any change in their output can be attributed primarily to thermal drift or similar non-mechanical influences.

In operation, the control electronics of the knee alignment measurement tool may continuously monitor the readings from the non-load-bearing sensors and use these readings to compensate the outputs of the load-bearing sensors 910, 914, 920, and 924. For example, a calibration algorithm may determine how much of the change in sensor output is due to thermal expansion and subtract this component from the total signal of the load-bearing elements, effectively normalizing the force readings to a reference condition. This compensation can be performed independently for the left and right sides by pairing each group of load-bearing sensors with its corresponding non-load-bearing sensor. As a result, the instrument can maintain high accuracy and stability of force measurement across a range of operating temperatures, temperature transients going from room to in vivo temperatures and over extended periods of intraoperative use, improving confidence in both total force values and medial-lateral balance readings.

In addition, the presence of multiple sensors on each side allows for more detailed mapping of the load distribution within each compartment if desired. The primary load-bearing sensors can be positioned to capture the main condylar contact regions, while the non-load-bearing sensors provide thermal reference signals at nearby locations, ensuring that the compensation remains spatially relevant. This multi-element configuration may also facilitate future software features, such as detection of off-center loading patterns within a single condyle or enhanced diagnostics for sensor integrity, thereby further increasing the robustness and clinical utility of the knee alignment measurement tool.

It is contemplated that any embodiment discussed in this specification can be implemented with respect to any method of the invention, and vice versa. It will be also understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. Incorporation by reference is limited such that no subject matter is incorporated that is contrary to the explicit disclosure herein, no claims included in the documents are incorporated by reference herein, and any definitions provided in the documents are not incorporated by reference herein unless expressly included herein.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12, 15, 20 or 25%.

All of the devices and/or methods disclosed and claimed herein can be made and executed without undue experimentation in light of the present disclosure. While the devices and methods of this invention have been described in terms of preferred embodiments, it will be apparent to those of skill in the art that variations may be applied to the devices and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A knee alignment measurement tool comprising:
a sensor assembly comprising, in turn, a rigid base supporting a left sensor and a right sensor positioned adjacent to the left sensor, and a cover sealed to and spaced apart from the rigid base, the cover comprises at least one thicker portion configured to transmit compression force applied on top thereof without affecting remaining areas of the cover, and
a display unit operatively connected to the sensor assembly and configured to power up, operatively control the left sensor and the right sensor, and display the readings therefrom,
wherein the knee alignment measurement tool is configured to provide feedback on compression forces between components of a knee replacement prosthesis and a balance of forces between a left condyle and a right condyle.

2. The knee alignment measurement instrument, as in claim 1, wherein the sensor assembly comprises a group of left sensors and a group of right sensors, at least one sensor of each group being configured as a primary load-bearing sensor and at least one sensor of each group being configured as a non-load-bearing sensor, the cover of the sensor assembly being configured such that no thicker portion is arranged above each non-load-bearing sensor so that no significant compressive load is transmitted thereto, and wherein control electronics are configured to monitor outputs of the non-load-bearing sensors as in situ reference signals indicative of thermal expansion and environmental effects, and to compensate outputs of the load-bearing sensors based on the reference signals so as to improve accuracy and stability of force measurement across changing operating temperatures.

3. The knee alignment tool, as in claim 1, wherein the thicker portion of the cover is surrounded by a thinner portion forming a living hinge around the thicker portion to allow the thicker portion to transmit compression force applied on top thereof to the left sensor or the right sensor without deforming and affecting remaining areas of the cover.

4. The knee alignment tool, as in claim 1, wherein the cover is spaced apart from the rigid base to form a predefined gap therebetween, so that the cover does not abut the rigid base during the compression of the sensor assembly.

5. A knee alignment measurement instrument comprising:
a knee alignment measurement tool of claim 1, further comprising a center post positioned between the left sensor and the right sensor and extending above the cover,
a femur-facing insert comprising a femoral surface configured to face a femur and to stay in contact therewith during a knee alignment measurement procedure, and
a shim configured to be inserted between the femur-facing insert and the knee alignment measurement tool while constrained by the center post thereof, wherein the shim is configured to change a distance between the femur-facing insert and the knee alignment measurement tool.

6. The knee alignment measurement instrument, as in claim 5, wherein the shim comprises a plurality of interchangeable shims of various thicknesses, thereby inserting one shim at a time defines the distance between the femur-facing insert and the knee alignment measurement tool to correspond to a thickness of the inserted shim of the plurality of interchangeable shims.

7. An inflatable shim for adjusting a distance between a femur and a tibia during a knee alignment measurement procedure, the inflatable shim is configured to form a stack with a femur-facing insert and a knee alignment measurement tool, the inflatable shim comprises an inflatable bladder and a distance sensor, wherein inflation of the inflatable bladder causes a measurable increase of a total height of the stack corresponding to a measurable increase of the distance between the femur and the tibia,
wherein the inflatable bladder comprises, in turn, an inflation port configured to ingress and egress air in or out of the inflatable bladder, causing a corresponding change in a height thereof.

8. The inflatable shim, as in claim 7, further comprising a rigid shim base having the inflatable bladder mounted thereon.

9. The inflatable shim, as in claim 8, wherein the rigid shim base is detachable from the inflatable bladder so as to facilitate replacement of the inflatable bladder and reuse of the rigid shim base.

10. A knee alignment measurement instrument comprising the inflatable shim, as in claim 7, wherein the knee alignment measurement tool is configured to be positioned between the inflatable shim and the femur-facing insert to form the stack of the inflatable shim, the knee alignment measurement tool, and the femur-facing insert.

11. A knee alignment measurement instrument comprising the inflatable shim, as in claim 8, wherein the knee alignment measurement tool comprises a center post extending therefrom, the inflatable shim is configured to be positioned at least partially around the center post and between the knee alignment measurement instrument and the femur-facing insert.

12. The knee alignment measurement instrument, as in claim 10, wherein the knee alignment measurement tool further comprises a sensor assembly comprising, in turn, a rigid base supporting a left sensor and a right sensor positioned adjacent to the left sensor, and a cover configured to transmit compression force applied on top thereof to individually the left sensor and the right sensor, and
a display unit operatively connected to the sensor assembly and the distance sensor and configured to power up, operatively control the left sensor, the right sensor, and the distance sensor, and display the readings therefrom,
wherein the knee alignment measurement tool is configured to provide feedback on compression forces between components of a knee replacement prosthesis, a balance of forces between a left condyle and a right condyle, and the distance between the femur and the tibia.

13. The inflatable shim, as in claim 7, wherein inflation of the inflatable bladder increases the total height of the stack from an additional 1 mm to an additional 8 mm.

14. The inflatable shim, as in claim 7, wherein the distance sensor is a capacitive distance-measuring sensor.

15. The inflatable shim, as in claim 7, wherein the distance sensor is a lidar sensor configured to measure the distance using a pulsed laser light.

16. The inflatable shim, as in claim 12, wherein the inflatable bladder comprises a left balloon and a right balloon.

17. The inflatable shim, as in claim 16, wherein when the inflatable shim is positioned between the knee alignment measurement tool and the femur-facing insert, the left balloon is shaped and aligned with the left sensor and the right balloon is shaped and aligned with the right sensor, thereby inflation of the inflatable bladder causes a corresponding transmission of compression forces from the left ballon to the left sensor and from the right balloon to the right sensor.

18. The inflatable shim, as in claim 16, wherein the left balloon and the right balloon each comprise or is operatively connected to a pressure sensor configured to measure the internal air pressure within the respective balloon, and wherein the pressure measured in the left balloon corresponds to the compressive force applied to the medial compartment of the knee joint and the pressure measured in the right balloon corresponds to the compressive force applied to the lateral compartment of the knee joint, thereby enabling the display unit to calculate both total joint force and medial-lateral force balance from the pressure readings of the left balloon and the right balloon without requiring separate solid-state force sensors within the sensor assembly.
